(19) United States
(12) Patent Application Publication
(10) Pub. No.: US 2023/0225991 A1
(43) Pub. Date: Jul. 20, 2023

(54) COMPOUNDS FOR USE IN THE TREATMENT OF ACUTE INTERMITTENT PORPHYRIA

(71) Applicant: VESTLANDETS INNOVASJONSSELSKAP AS, Bergen (NO)

(72) Inventors: Aurora MARTINEZ, Bergen (NO); Helene Bustad JOHANNESSEN, Bergen (NO); Juha Pekka KALLIO, Bergen (NO); Jarl UNDERHAUG, Bergen (NO); Aasne Karine AARSAND, Bergen (NO); Marta VORLAND, Bergen (NO); Sverre SANDBERG, Bergen (NO); Karen TOSKA, Bergen (NO); Caroline SCHMITT, Paris (FR); Laurent GOUYA, Paris (FR); Lars SKJÆVEN, Bergen (NO); James HITCHIN, Nottingham (GB); Sarah MAJOR, Nottingham (GB)

(21) Appl. No.: 17/778,442

(22) PCT Filed: Nov. 20, 2020
(86) PCT No.: PCT/GB2020/052975

§ 371 Date: May 20, 2022

(30) Foreign Application Priority Data
Nov. 21, 2019 (GB) 1916983.8

(51) Int. Cl. A61K 31/12; A61K 31/44; A61K 31/065; A61K 31/04; A61K 31/167; A61K 31/11; A61K 31/4025; A61K 31/05

(52) U.S. Cl. CPC A61K 31/12; A61K 31/44; A61K 31/065; A61K 31/04; A61K 31/167; A61K 31/11; A61K 31/4025; A61K 31/05

(57) ABSTRACT

The invention provides compounds of formula (I), their pharmaceutically acceptable salts and prodrugs thereof for use in preventing, inhibiting or treating a disease caused by a mutation in the gene coding for hydroxymethylbilane synthase, in particular for preventing, inhibiting or treating acute intermittent porphyria: (I) wherein: A is selected from N and CR¹⁰ (wherein R¹⁰ is H, —NO₂, C₁₋₆ haloalkyl or —C(O)R¹⁷ in which R¹⁷ is H or C₁₋₆ alkyl); Z is selected from N and CR⁹ (wherein R⁹ is H, halogen (e.g. F, Cl, Br or I) or —OR¹⁶ in which R¹⁶ is H, C₁₋₆ haloalkyl, or optionally substituted C₁₋₆ alkyl); L is selected from —CH₂—, —C(O)—, —CH(OH)—, —C(O)—NR′—, and —NR′—C(O)— (wherein R′ is H or C₁₋₃ alkyl, e.g. —CH₃); R¹ is H; R² is selected from H, halogen (e.g. F, Cl, Br or I), —NR¹¹R¹² (wherein R¹¹ and R¹² are independently selected from H and C₁₋₆ alkyl or, together with the nitrogen atom to which they are attached, form a 5- or 6-membered saturated ring), and —OR13 (wherein R¹³ is H or C₁₋₆ alkyl); R³ is selected from H, —CH²OH and —C(O)R¹⁴ (wherein R¹⁴ is H or C₁₋₆ alkyl); R⁴ is selected from H, halogen (e.g. F, Cl, Br or I) and —OR¹⁵ (where R¹⁵ is H or C₁₋₆ alkyl); R⁵ is selected from H and C₁₋₆ alkyl; R⁶ is selected from H, —NO₂ and halogen (e.g. F, Cl, Br or I); R⁷ is H; and R⁸ is selected from H, C₁₋₆ alkyl, and halogen (e.g. F, Cl, Br or I); or wherein: R⁷ and R⁸ together with the intervening ring carbon atoms form an unsaturated ring, preferably an aryl ring.

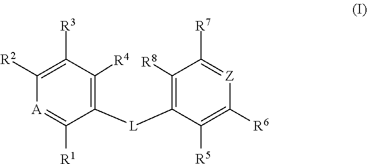

### FIELD OF THE INVENTION

The present invention relates to the use of compounds in preventing, inhibiting or treating diseases caused by mutations in the gene coding for hydroxymethylbilane synthase (HMBS, EC 2.5.1.61), an enzyme involved in the heme biosynthetic pathway. More specifically, the invention relates to the use of such compounds in alleviating or preventing the symptoms of acute intermittent porphyria (AIP).

The invention further relates to certain novel compounds, to pharmaceutical compositions containing them, and to their use in such treatment.

### BACKGROUND OF THE INVENTION

Acute intermittent porphyria (AIP) is an autosomal dominantly inherited inborn error of metabolism caused by mutations in the gene coding for the third enzyme of the heme biosynthetic pathway, i.e. hydroxymethylbilane synthase (HMBS, EC 2.5.1.61). To date, more than 420 different mutations in the HMBS gene have been reported, including both catalytically and/or conformationally deleterious mutations. The prevalence of AIP is about 1 in 20,000-100,000, depending on the ethnic group. However, the overall penetrance is 0.5-1% in the general population and 10-20% within families. Factors such as hormonal changes, low carbohydrate intake, alcohol or porphyrinogenic drugs activate the expression of hepatic δ-aminolevulinic acid (ALA) synthase 1 (ALAS1). Elevated ALAS1 together with diminished HMBS activity lead to accumulation of the heme precursors ALA and porphobilinogen (PBG). PBG and, in particular, ALA are believed to be toxic metabolites related to the neuropathy of the disease and to trigger the acute attacks. In addition, PBG at high concentrations may further decrease HMBS activity. The attacks are unspecific with common symptoms such as abdominal pain, nausea, tachycardia and hypertension, in addition to various neurological and psychiatric symptoms. More severe symptoms such as acute psychosis and potentially life-threatening symptoms—paralysis and coma—may also occur. AIP patients have a higher risk of developing hepatocellular carcinoma, hypertension and kidney failure.

Intravenous administration of human hemin is the established treatment for severe and recurrent AIP attacks, providing exogenous heme that down-regulates ALAS1 expression. However, repeated therapy can be associated with reduced effectiveness and may also give a chronic activation of heme oxygenase 1 (HO1) expression that will trigger ALAS1 and subsequently recurrent attacks. Liver transplantation is today the only curative alternative for chronically ill patients. Although several therapeutic options are under investigation, including hepatocyte transplantation, liver-directed gene therapy, and subcutaneous ALAS1 RNAi therapy for the treatment of hepatic porphyrias using circulating RNA quantification, there is still a need for effective mechanism-based pharmacotherapies. These have the potential to provide a non-invasive, oral treatment that could work prophylactically, as well as a specific medication for use during an acute attack.

Pharmacological chaperones (PCs) are small molecular weight compounds that specifically target and interact with unstable and incorrectly folded proteins. PC binding stabilizes the target protein protecting it from early degradation, thus increasing its half-life and enhancing its cellular activity. PC therapy has been demonstrated as a potential treatment for protein misfolding diseases such as cystic fibrosis, phenylketonuria, lysosomal storage disorders, and congenital erythropoietic porphyria. Residual enzymatic activity is required for the PCs to enhance the activity of conformational mutations resulting in unstable and misfolded enzyme.

The focus for the PC-discovery in the above-mentioned recessive genetic diseases has been the development of PCs targeting either one particularly common mutation in the target protein or a range of responsive mutations. However, AIP presents as a model disorder for autosomal dominantly inherited diseases, where fully functional wild-type (WT) HMBS is expressed from only one allele and provides ˜50% of normal enzymatic activity. This is seemingly enough to maintain normal cellular metabolism (Badminton et al., Journal of inherited metabolic disease, 2005; 28(3): 277-86), although once the heme synthesis is challenged, the amount of functional gene product is unable to compensate for the allele that holds the HMBS-mutation. Relevantly for AIP and other autosomal dominantly inherited disorders, PCs have demonstrated value in increasing the stability of WT enzymes in vitro and in vivo (Jorge-Finnigan et al, Human molecular genetics, 2013; 22(18):3680-9).

### SUMMARY OF THE INVENTION

The inventors have now identified compounds which are capable of stabilizing WT-HMBS and thus have potential in the development of a PC therapy for AIP independent of the patient's mutation. Such compounds may be used both curatively for acute attacks and prophylactically to impede recurrent acute attacks.

The inventors' findings have been validated in vitro by analyzing the effect of the compounds on the conformational and kinetic stability of purified recombinant WT-HMBS. As described herein, their pharmacological chaperone potential in human hepatoma HepG2 cells over-expressing WT-HMBS, and in vivo using an Hmbs-deficient T1/T2^{-/-} mouse model has also been evaluated. The Hmbs-deficient mice exhibits ˜30% of normal hepatic activity, and is compound heterozygous of one null allele and one low-expressed normal allele.

In one aspect, the invention relates to a compound of formula (I), or a pharmaceutically acceptable salt or prodrug thereof for use in preventing, inhibiting or treating a disease caused by a mutation in the gene coding for hydroxymethylbilane synthase (HMBS), in particular for preventing, inhibiting or treating acute intermittent porphyria:

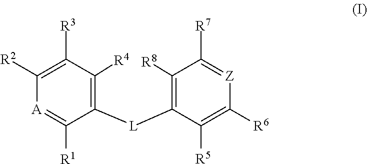

wherein:
A is selected from N and CR¹⁰ (wherein R¹⁰ is H, —NO₂, C₁₋₆ haloalkyl or —C(O)R¹⁷ in which R¹⁷ is H or C₁₋₆ alkyl);
Z is selected from N and CR⁹ (wherein R⁹ is H, halogen (e.g. F, Cl, Br or I) or —OR¹⁶ in which R¹⁶ is H, C₁₋₆ haloalkyl, or optionally substituted C₁₋₆ alkyl);
L is selected from —CH₂—, —C(O)—, —CH(OH)—, —C(O)—NR′—, and —NR′—C(O)— (wherein R′ is H or C₁₋₃ alkyl, e.g. —CH₃);
R¹ is H;
R² is selected from H, halogen (e.g. F, Cl, Br or I), —NR¹¹R¹² (wherein R¹¹ and R¹² are independently selected from H and C₁₋₆ alkyl or, together with the nitrogen atom to which they are attached, form a 5- or 6-membered saturated ring), and —OR¹³ (wherein R¹³ is H or C₁₋₆ alkyl);
R³ is selected from H, —CH₂OH and —C(O)R¹⁴ (wherein R¹⁴ is H or C₁₋₆ alkyl);
R⁴ is selected from H, halogen (e.g. F, Cl, Br or I) and —OR¹⁵ (where R¹⁵ is H or C₁₋₆ alkyl);
R⁵ is selected from H and C₁₋₆ alkyl;
R⁶ is selected from H, —NO₂ and halogen (e.g. F, Cl, Br or I);
R⁷ is H; and
R⁸ is selected from H, C₁₋₆ alkyl, and halogen (e.g. F, Cl, Br or I);
or wherein:
   R⁷ and R⁸ together with the intervening ring carbon atoms form an unsaturated ring, preferably an aryl ring.

In a further aspect, the invention relates to a compound of formula (I), or a pharmaceutically acceptable salt or prodrug thereof, for use in therapy or for use as a medicament.

In another aspect, the invention relates to a pharmaceutical composition comprising a compound of formula (I), or a pharmaceutically acceptable salt or prodrug thereof, together with one or more pharmaceutically acceptable carriers, excipients or diluents.

In a further aspect the invention relates to the use of a compound of formula (I), or a pharmaceutically acceptable salt or prodrug thereof, in the manufacture of a medicament for use in the prevention, treatment or inhibition of a disease caused by a mutation in the gene coding for hydroxymethylbilane synthase, in particular for the prevention, inhibition or treatment of acute intermittent porphyria.

In another aspect, the invention relates to a method of prevention or treatment of a disease caused by a mutation in the gene coding for hydroxymethylbilane synthase, in particular a method of prevention or treatment of acute intermittent porphyria, said method comprising the step of administering to a patient in need thereof (e.g. a human subject) a pharmaceutically effective amount of a compound of formula (I), or a pharmaceutically acceptable salt or prodrug thereof.

In another aspect the invention relates to certain novel compounds of formula (VIII), their pharmaceutically acceptable salts, and prodrugs:

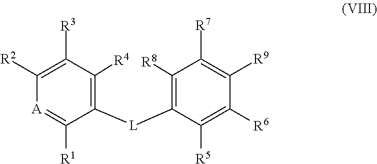

wherein:
A is selected from N and CR¹⁰ (wherein R¹⁰ is —NO₂, C₁₋₆ haloalkyl or —C(O)R¹⁷ in which R¹⁷ is H or C₁₋₆ alkyl);
L is selected from —CH₂—, —C(O)—, —CH(OH)—, —C(O)—NR′—, and —NR′—C(O)— (wherein R′ is H or C₁₋₃ alkyl, e.g. —CH₃);
R¹ is H;
R² is selected from halogen (e.g. F, Cl, Br or I), —NR¹¹R¹² (where R¹¹ and R¹² are independently selected from H and C₁₋₆ alkyl or, together with the nitrogen atom to which they are attached, form a 5- or 6-membered saturated ring), and —OR¹³ (wherein R¹³ is H or C₁₋₆ alkyl);
R³ is H;
R⁴ is H;
R⁵ is selected from H and C₁₋₆ alkyl;
R⁶ is H;
R⁷ is H;
R⁸ is selected from H, C₁₋₆ alkyl, and halogen (e.g. F, Cl, Br or I); and
R⁹ is —OR¹⁶ (where R¹⁶ is H, C₁₋₆ haloalkyl, or optionally substituted C₁₋₆ alkyl); with the proviso that the compound is other than:
(4-chloro-3-nitrophenyl)(phenyl)methanone or
(4-chloro-3-nitrophenyl)(4-methoxyphenyl)methanone.

### DETAILED DESCRIPTION OF THE INVENTION

### Definitions

As used herein, the term “alkyl” refers to a saturated hydrocarbon group and is intended to cover both straight-chained and branched alkyl groups. Examples of such groups include methyl, ethyl, n-propyl, iso-propyl, n-butyl, tert-butyl, sec-butyl, n-pentyl, iso-pentyl, neo-pentyl, n-hexyl, 2-methylbutyl, 2-methylpentyl, 2-ethylbutyl, 3-methylpentyl, and 4-methylpentyl. An alkyl group preferably contains from 1-6 carbon atoms, more preferably 1-4 carbon atoms, e.g. 1-3 carbon atoms. The term “alkyl” group also includes any saturated hydrocarbon group in which one or more (e.g. all) hydrogen atoms are replaced with deuterium. Examples of such groups include —CD₃, —CD₂CD₃, —CD₂CD₂CD₃, —CD(CD₃)CD₃, etc.

The term “halogen” or “halogen atom” as used herein refers to —F, —Cl, —Br or —I.

The term “haloalkyl” refers to an alkyl group as defined herein in which at least one of the hydrogen atoms of the alkyl group is replaced by a halogen atom, preferably F, Cl or Br. Examples of such groups include —CH₂F, —CHF₂, —CF₃, —CCl₃, —CHCl₂, —CH₂CF₃, etc.

The term “aryl” as used herein refers to aromatic ring systems. Such ring systems may be monocyclic or bicyclic and contain at least one unsaturated aromatic ring. Where these contain bicyclic rings, these may be fused. Preferably such systems contain from 6-20 carbon atoms, e.g. either 6 or 10 carbon atoms. Examples of such groups include phenyl, 1-napthyl and 2-napthyl. A preferred aryl group is phenyl.

The term “optionally substituted” as used herein refers to the presence of one or more substituents. Where more than one substituent group is present, these may be the same or different. Suitable substituents include hydroxy, amino (e.g. —NR′R″ in which R′ and R″ are independently selected from H and C₁₋₆ alkyl (e.g. C₁₋₃ alkyl) or, together with the nitrogen atom to which they are attached, form a 5- or 6-membered saturated ring), cyano, nitro groups, or halogen atoms (e.g. F, Cl or Br).

Unless otherwise stated, all substituents are independent of one another.

The term “pharmaceutically acceptable salt” as used herein refers to any pharmaceutically acceptable organic or inorganic salt of any of the compounds herein described. A pharmaceutically acceptable salt may include one or more additional molecules such as counter-ions. The counter-ions may be any organic or inorganic group which stabilizes the charge on the parent compound. If the compound of the invention is a base, a suitable pharmaceutically acceptable salt may be prepared by reaction of the free base with an organic or inorganic acid. If the compound of the invention is an acid, a suitable pharmaceutically acceptable salt may be prepared by reaction of the free acid with an organic or inorganic base. Non-limiting examples of suitable salts are described herein.

The term “pharmaceutically acceptable” means that the compound or composition is chemically and/or toxicologically compatible with other components of the formulation or with the patient to be treated.

By “a pharmaceutical composition” is meant a composition in any form suitable to be used for a medical purpose.

The term “prodrug” refers to a derivative of an active compound which undergoes a transformation under the conditions of use, for example within the body, to release an active drug. A prodrug may, but need not necessarily, be pharmacologically inactive until converted into the active drug. As used herein, the term “prodrug” extends to any compound which under physiological conditions is converted into any of the active compounds herein described. Suitable prodrugs include compounds which are hydrolysed under physiological conditions to the desired molecule.

Prodrugs may typically be obtained by masking one or more functional groups in the parent molecule which are considered to be, at least in part, required for activity using a suitable progroup. By “progroup” as used herein is meant a group which is used to mask a functional group within an active drug and which undergoes a transformation, such as cleavage, under the specified conditions of use (e.g. administration to the body) to release a functional group and hence provide the active drug. Progroups are typically linked to the functional group of the active drug via a bond or bonds that are cleavable under the conditions of use, e.g. in vivo. Cleavage of the progroup may occur spontaneously under the conditions of use, for example by way of hydrolysis, or it may be catalysed or induced by other physical or chemical means, e.g. by an enzyme, by exposure to light, by exposure to a change in temperature, or to a change in pH, etc. Where cleavage is induced by other physical or chemical means, these may be endogenous to the conditions of use, for example pH conditions at a target site, or these may be supplied exogenously.

As used herein, “treatment” includes any therapeutic application that can benefit a human or non-human animal subject (e.g. a human) and is intended to refer to the reduction, alleviation or elimination, preferably to normal levels, of one or more of the symptoms of the disease, disorder or condition which is being treated relative to the symptoms prior to treatment.

As used herein, “prevention” refers to absolute prevention, i.e. maintenance of normal levels with reference to the extent or appearance of a particular symptom of the disease, disorder, or condition, or reduction or alleviation of the extent or timing (e.g. delaying) of the onset of that symptom.

As used herein, a “pharmaceutically effective amount” relates to an amount that will lead to the desired pharmacological and/or therapeutic effect, i.e. an amount of the agent which is effective to achieve its intended purpose. While individual patient needs may vary, determination of optimal ranges for effective amounts of the active agent is within the capability of one skilled in the art. Generally, the dosage regimen for treating a disease or condition with any of the compounds described herein is selected in accordance with a variety of factors including the nature of the medical condition and its severity.

As used herein, “hydroxymethylbilane synthase” refers to an enzyme which is involved in the third step of the heme biosynthetic pathway and which catalyses the condensation of four porphobilinogen (PBG) molecules into hydroxymethylbilane. In particular, it refers to an enzyme having the classification EC 2.5.1.61.

The inventors have now found that the compounds herein described can stabilize WT-HMBS. This discovery leads to the use of the compounds to treat or prevent conditions or diseases in subjects, particularly in humans, which are mediated by the activity of WT-HMBS. As stabilizers of WT-HMBS the compounds herein described are particularly suitable for preventing or treating acute intermittent porphyria.

In one aspect, the invention provides a compound of formula (I), or a pharmaceutically acceptable salt or prodrug thereof for use in preventing, inhibiting or treating a disease caused by a mutation in the gene coding for hydroxymethylbilane synthase, in particular for preventing, inhibiting or treating acute intermittent porphyria:

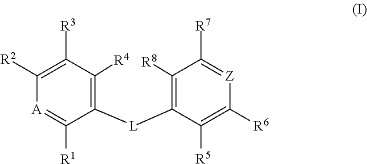

wherein:
A is selected from N and CR¹⁰ (wherein R¹⁰ is H, —NO₂, C₁₋₆ haloalkyl or —C(O)R¹⁷ in which R¹⁷ is H or C₁₋₆ alkyl);
Z is selected from N and CR⁹ (wherein R⁹ is H, halogen (e.g. F, Cl, Br or I) or —OR¹⁶ in which R¹⁶ is H, C₁₋₆ haloalkyl, or optionally substituted C₁₋₆ alkyl);
L is selected from —CH₂—, —C(O)—, —CH(OH)—, —C(O)—NR′—, and —NR′—C(O)— (wherein R′ is H or C₁₋₃ alkyl, e.g. —CH₃);
R¹ is H;
R² is selected from H, halogen (e.g. F, Cl, Br or I), —NR¹¹R¹² (wherein R¹¹ and R¹² are independently selected from H and C₁₋₆ alkyl or, together with the nitrogen atom to which they are attached, form a 5- or 6-membered saturated ring), and —OR¹³ (wherein R¹³ is H or C₁₋₆ alkyl);
R³ is selected from H, —CH₂OH and —C(O)R¹⁴ (wherein R¹⁴ is H or C₁₋₆ alkyl);
R⁴ is selected from H, halogen (e.g. F, Cl, Br or I) and —OR¹⁵ (where R¹⁵ is H or C₁₋₆ alkyl);
R⁵ is selected from H and C₁₋₆ alkyl;
R⁶ is selected from H, —NO₂ and halogen (e.g. F, Cl, Br or I);
R⁷ is H; and
R⁸ is selected from H, C₁₋₆ alkyl, and halogen (e.g. F, Cl, Br or I);
or wherein:
   R⁷ and R⁸ together with the intervening ring carbon atoms form an unsaturated ring, preferably an aryl ring.

In one embodiment, A is CR¹⁰ in which R¹⁰ is as herein defined. In one set of embodiments, R¹⁰ is —NO₂ or C₁₋₆ haloalkyl (preferably C₁₋₃ haloalkyl, e.g. —CF₃). In one embodiment, R¹⁰ is —NO₂ or —CF₃.

In one embodiment, Z is CR⁹ in which R⁹ is as herein defined. In one set of embodiments, R⁹ is H, halogen (preferably F, Cl, or Br, e.g. Cl) or —OR¹⁶ in which R¹⁶ is H or C₁₋₆ alkyl, preferably C₁₋₃ alkyl, e.g. —CH₃). In one embodiment, R⁹ is selected from H, halogen (e.g. F, Cl, or Br, preferably Cl) and —OR¹⁶ (where R¹⁶ is H, —CF₃ or —CH₃). In another embodiment, R⁹ is H, Cl, —OCF₃ or —OCH₃. In a further embodiment R⁹ is H or —OCF₃.

In one embodiment, L is selected from —CH₂—, —C(O)—, —CH(OH)—, and —C(O)—N(CH₃)—. In another embodiment, L is either —CH₂— or —C(O)—.

In one embodiment, R² is selected from H, halogen (preferably F, Cl, or Br, e.g. Cl), and —OR¹³ (wherein R¹³ is H or C₁₋₆ alkyl, preferably C₁₋₃ alkyl, e.g. —CH₃). In another embodiment, R² is selected from H, —OCH₃, —OH and Cl. In one embodiment R² is either OH or Cl.

In one embodiment, R³ is selected from H and —C(O)R¹⁴ (wherein R¹⁴ is H or C₁₋₆ alkyl, preferably C₁₋₃ alkyl, e.g. —CH₃). In another embodiment, R³ is H or —C(O)H.

In one embodiment, R⁴ is selected from H, —OH and halogen (preferably F, Cl, or Br, e.g. Cl).

In one embodiment, R⁵ is H or C₁₋₃ alkyl, e.g. —CH₃.

In one embodiment, R⁶ is selected from H and halogen (preferably F, Cl, or Br, e.g. Cl). In one embodiment, R⁶ is selected from H and Cl.

In one embodiment, R⁸ is selected from H, halogen (preferably F, Cl, or Br, e.g. Cl) and C₁₋₃ alkyl, e.g. —CH₃. In another embodiment, R⁸ is selected from H and Cl.

In one embodiment of formula (I), R⁷ and R⁸ together with the intervening ring carbon atoms may form an unsaturated ring, for example a 5- or 6-membered unsaturated carbocyclic ring. The carbocyclic ring may be aromatic or non-aromatic. In one embodiment, R⁷ and R⁸ together with the intervening ring carbon atoms form an aryl ring, for example an optionally substituted phenyl ring. In one embodiment, the aryl ring may be unsubstituted.

In one embodiment, the compounds for use in the invention are those of formula (II), their pharmaceutically acceptable salts, or prodrugs thereof:

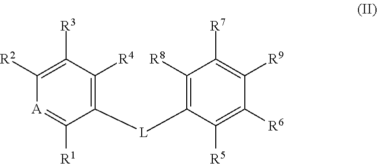

wherein A, L and R¹ to R⁹ are as defined herein.

In another embodiment, the compounds for use in the invention are those of formula (III), their pharmaceutically acceptable salts, or prodrugs thereof:

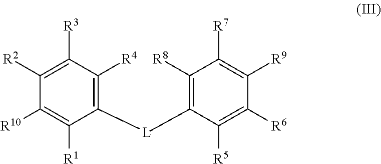

wherein L and R¹ to R¹⁰ are as defined herein.

In another embodiment, the compounds for use in the invention are those of formula (IV), their pharmaceutically acceptable salts, or prodrugs thereof:

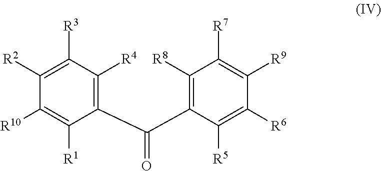

wherein R¹ to R¹⁰ are as defined herein.

In another embodiment, the compounds for use in the invention are those of formula (IVa), their pharmaceutically acceptable salts, or prodrugs thereof:

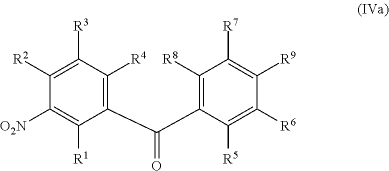

wherein R¹ to R⁹ are as defined herein.

In another embodiment, the compounds for use in the invention are those of formula (IVb), their pharmaceutically acceptable salts, or prodrugs thereof:

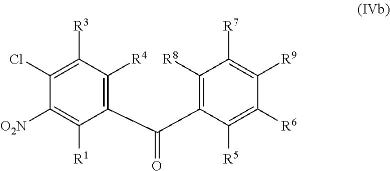

wherein R¹ and R³ to R⁹ are as defined herein.

In one embodiment, in the compounds of formula (IV) or formula (IVa), R² is selected from H, —OCH₃ and Cl.

In one embodiment, in the compounds of formula (IV), formula (IVa) or formula (IVb), R³ is H. In one embodiment, R⁴ is H. In one embodiment, R⁶ is H. In one embodiment, R⁹ is selected from H, Cl and —OCF₃.

In another embodiment, the compounds for use in the invention are those of formula (V), their pharmaceutically acceptable salts, or prodrugs thereof:

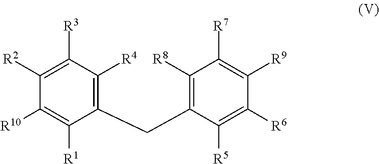

wherein R¹ to R¹⁰ are as defined herein.

In another embodiment, the compounds for use in the invention are those of formula (Va), their pharmaceutically acceptable salts, or prodrugs thereof:

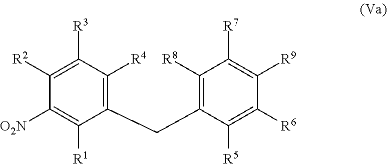

wherein R¹ to R⁹ are as defined herein.

In another embodiment, the compounds for use in the invention are those of formula (Vb), their pharmaceutically acceptable salts, or prodrugs thereof:

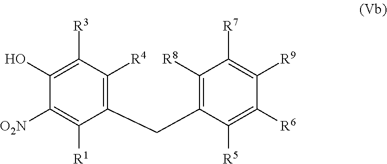

wherein R¹ and R³ to R⁹ are as defined herein.

In another embodiment, the compounds for use in the invention are those of formula (VI), their pharmaceutically acceptable salts, or prodrugs thereof:

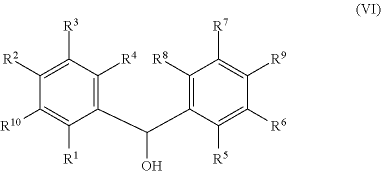

wherein R¹ to R¹⁰ are as defined herein.

In another embodiment, the compounds for use in the invention are those of formula (VII), their pharmaceutically acceptable salts, or prodrugs thereof:

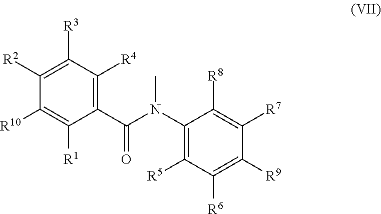

wherein R¹ to R¹⁰ are as defined herein.

Examples of compounds for use in the invention include, but are not limited to, the following and their pharmaceutically acceptable salts and prodrugs thereof:

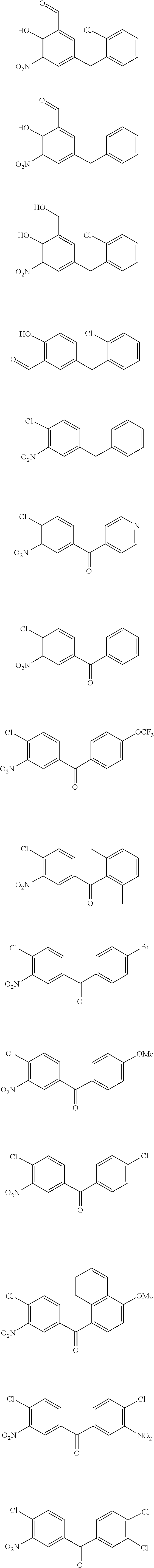

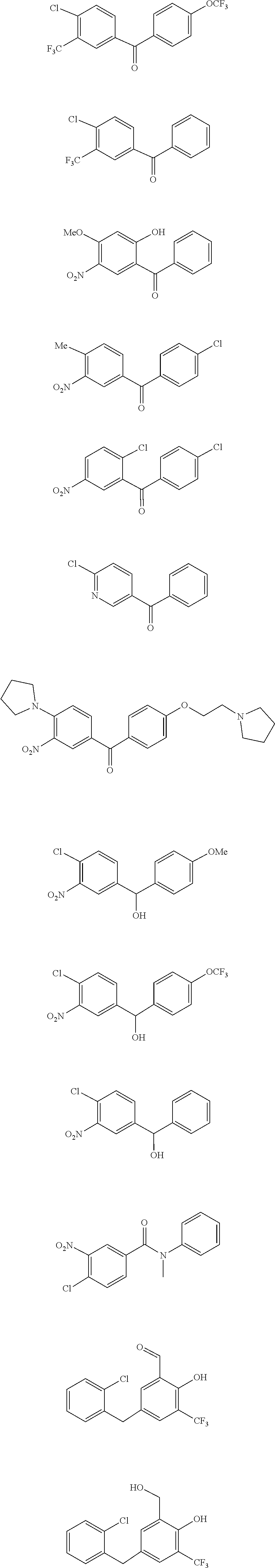

Preferred compounds for use in the invention are the following, their pharmaceutically acceptable salts and prodrugs thereof:

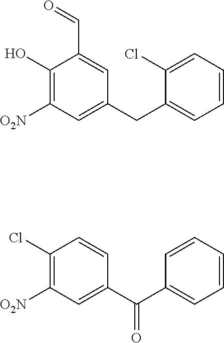 [SMILES: O=Cc1cc(Cc2ccccc2Cl)cc([N+](=O)[O-])c1O ; O=C(c1ccccc1)c1ccc(Cl)c([N+](=O)[O-])c1]

Certain compounds described herein are novel and these form a further aspect of the invention. Thus, in a further aspect, the present invention provides compounds of formula VIII, their pharmaceutically acceptable salts, and prodrugs thereof:

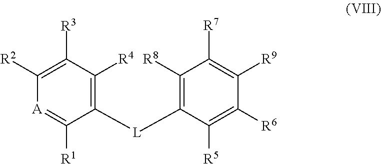

wherein:
A is selected from N and CR¹⁰ (wherein R¹⁰ is —NO₂, C₁₋₆ haloalkyl or —C(O)R¹⁷ in which R¹⁷ is H or C₁₋₆ alkyl);
L is selected from —CH₂—, —C(O)—, —CH(OH)—, —C(O)—NR′—, and —NR′—C(O)— (wherein R′ is H or C₁₋₃ alkyl, e.g. —CH₃);
R¹ is H;
R² is selected from halogen (e.g. F, Cl, Br or I), —NR¹¹R¹² (where R¹¹ and R¹² are independently selected from H and C₁₋₆ alkyl or, together with the nitrogen atom to which they are attached, form a 5- or 6-membered saturated ring), and —OR¹³ (wherein R¹³ is H or C₁₋₆ alkyl);
R³ is H;
R⁴ is H;
R⁵ is selected from H and C₁₋₆ alkyl;
R⁶ is H;
R⁷ is H;
R⁸ is selected from H, C₁₋₆ alkyl, and halogen (e.g. F, Cl, Br or I); and
R⁹ is —OR¹⁶ (where R¹⁶ is H, C₁₋₆ haloalkyl, or optionally substituted C₁₋₆ alkyl); with the proviso that the compound is other than:
(4-chloro-3-nitrophenyl)(phenyl)methanone or
(4-chloro-3-nitrophenyl)(4-methoxyphenyl)methanone.

In one embodiment of formula (VIII), A is selected from N and CR¹⁰ (wherein R¹⁰ is selected from —NO₂, —CF₃ and —C(O)H).

In one embodiment of formula (VIII), R² is selected from halogen (preferably F, Cl or Br, e.g. Cl), 1-pyrrolidinyl, and —OH.

In one embodiment of formula (VIII), R⁵ is selected from H and C₁₋₃ alkyl (e.g. —CH₃).

In one embodiment of formula (VIII), R⁸ is selected from H and C₁₋₃ alkyl (e.g. —CH₃).

In one embodiment of formula (VIII), R⁹ is —OR¹⁶ in which R¹⁶ is —CF₃, C₁₋₃ alkyl (e.g. —CH₃), or a group of the formula:

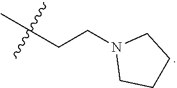 [SMILES: *CCN1CCCC1]

In a further aspect, the invention provides the following compounds, their pharmaceutically acceptable salts, and prodrugs thereof:

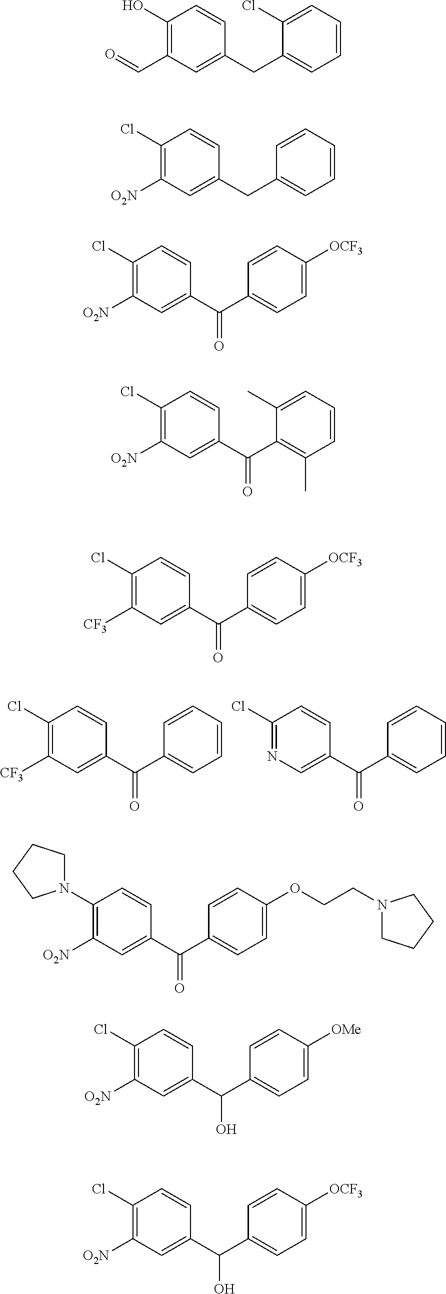

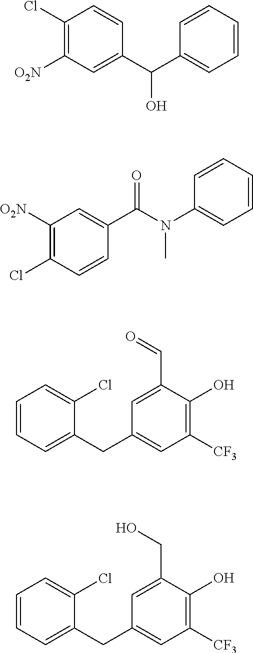 [SMILES: O=[N+]([O-])c1cc(C(O)c2ccccc2)ccc1Cl ; CN(C(=O)c1ccc(Cl)c([N+](=O)[O-])c1)c1ccccc1 ; O=Cc1cc(Cc2ccccc2Cl)cc(C(F)(F)F)c1O ; OCc1cc(Cc2ccccc2Cl)cc(C(F)(F)F)c1O]

The compounds for use in the invention may be provided in the form of a salt, particularly a pharmaceutically acceptable salt with an inorganic or organic acid or base. Acids which may be used for this purpose include hydrochloric acid, hydrobromic acid, sulfuric acid, sulfonic acid, methanesulfonic acid, phosphoric acid, fumaric acid, succinic acid, lactic acid, citric acid, tartaric acid, maleic acid, acetic acid, trifluoroacetic acid and ascorbic acid. Bases which may be suitable for this purpose include alkali and alkaline earth metal hydroxides, e.g. sodium hydroxide, potassium hydroxide or cesium hydroxide, ammonia and organic amines such as diethylamine, triethylamine, ethanolamine, diethanolamine, cyclohexylamine and dicyclohexylamine. Procedures for salt formation are conventional in the art.

In addition, any of the compounds described herein may be provided in the form of a prodrug. A prodrug is a compound which may have little or no pharmacological activity itself, but when such compound is administered into or onto the body of a patient it is converted into a compound having the desired activity.

Prodrugs may be obtained by masking one or more functional groups in the parent molecule using a progroup. A wide variety of progroups suitable for masking functional groups in active compounds to provide prodrugs are well known in the art. For example, a hydroxy functional group may be masked as an ester, a phosphate ester, or a sulfonate ester which may be hydrolyzed in vivo to provide the parent hydroxy group. Other examples of suitable progroups will be apparent to those of skill in the art.

The compounds for use in the invention are either known in the art, or can be prepared by methods known to those skilled in the art using readily available starting materials. A number of the compounds for use in the invention are commercially available from sources such as Vitas-M Laboratory Ltd and Alinda Chemical, Ltd.

Any of the compounds herein described which are not known in the art may be prepared from readily available starting materials using synthetic methods known in the art such as those described in known textbooks, for example, in Advanced Organic Chemistry (March, Wiley Interscience, 5^{th} Ed. 2001) or Advanced Organic Chemistry (Carey and Sundberg, KA/PP, 4^{th} Ed. 2001). For example, these may be made by Friedel-Crafts Acylation.

The following schemes show general methods for preparing the compounds herein described and key intermediates. Such methods form a further aspect of the invention. The compounds used as starting materials are either known from the literature or may be commercially available. Alternatively, these may readily be obtained by methods known from the literature. As will be understood, other synthetic routes may be used to prepare the compounds using different starting materials, different reagents and/or different reaction conditions. A more detailed description of how to prepare the compounds in accordance with the invention is found in the Examples.

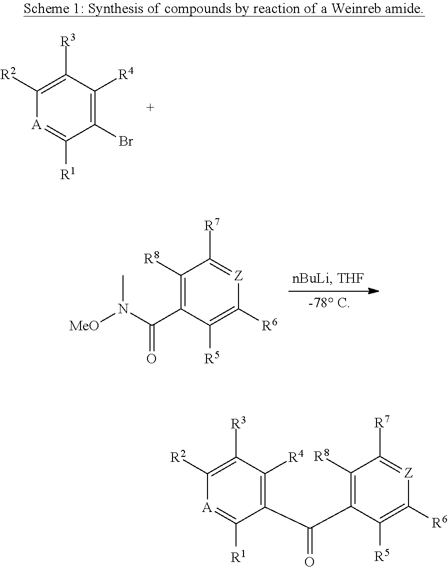

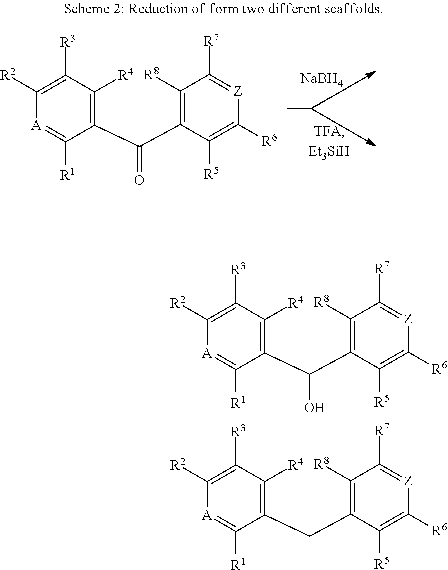

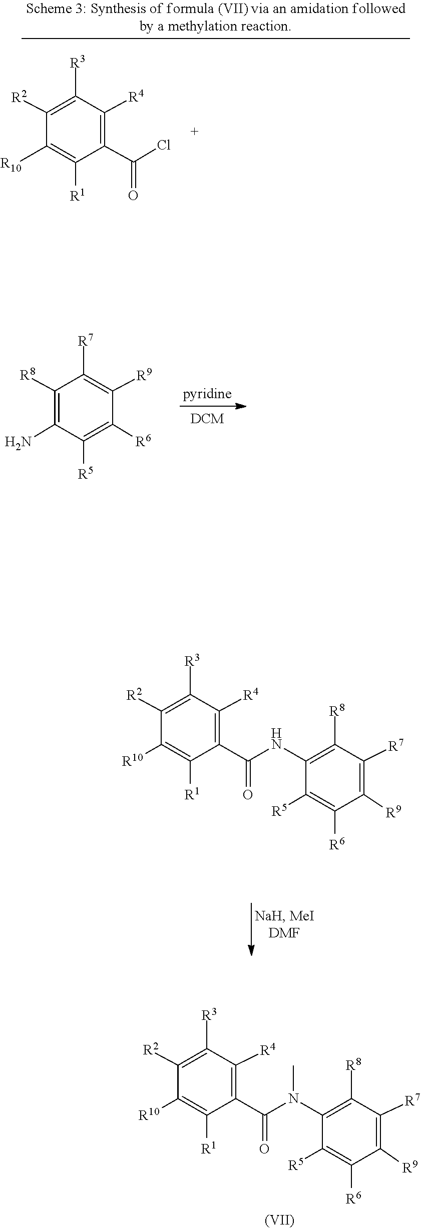

The compounds herein described have valuable pharmacological properties, particularly a stabilizing effect on WT-HMBS. In view of their ability to stabilize WT-HMBS, these are suitable for the prevention, inhibition or treatment of any condition or disease which is associated with a reduction in the activity of WT-HMBS. More generally, they are able to prevent, inhibit or treat conditions or diseases caused by a mutation in the gene coding for hydroxymethylbilane synthase (HMBS).

In particular, the compounds herein described are suitable for the inhibition, treatment or prevention of acute intermittent porphyria (AIP). For example, these may be used to reduce the frequency of recurrent acute attacks or prophylactically to prevent such attacks. Alternatively, these may be used therapeutically during an acute attack to avoid or reduce the symptoms of AIP.

In one embodiment, the compounds herein described may be used to treat the symptoms of an AIP attack. Symptoms of AIP may include, but are not limited to, any of the following: abdominal pain, urinary signs and symptoms (e.g. painful urination, urinary retention, urinary incontinence and dark urine), psychiatric signs and symptoms (e.g. anxiety, paranoia, irritability, delusions, hallucinations, confusion and depression), increased activity of the sympathetic nervous system (e.g. tachycardia, hypertension, palpitations, orthostatic hypotension, sweating, restlessness and tremor), and neurological signs and symptoms (e.g. seizures, peripheral neuropathy, abnormal sensations, chest pain, leg pain, back pain or headache and coma). Further symptoms may include nausea, vomiting, constipation, diarrhea, proximal muscle weakness, muscle pain, tingling, numbness, weakness, paralysis and muscle weakness.

Patients suffering from AIP have an increased risk of developing various other conditions such as hepatocellular carcinoma, melanoma, lymphoma, chronic hypertension, chronic kidney disease and chronic pain. In one embodiment, the compounds herein described may be used to prevent any such condition which may arise from ATP.

For use in a therapeutic or prophylactic treatment, the compounds herein described will typically be formulated as a pharmaceutical formulation. In a further aspect, the invention thus provides a pharmaceutical composition comprising a compound herein described, together with one or more pharmaceutically acceptable carriers, excipients or diluents.

Acceptable carriers, excipients and diluents for therapeutic use are well known in the art and can be selected with regard to the intended route of administration and standard pharmaceutical practice. Examples include binders, lubricants, suspending agents, coating agents, solubilizing agents, preserving agents, wetting agents, emulsifiers, surfactants, sweeteners, colorants, flavoring agents, antioxidants, odorants, buffers, stabilizing agents and/or salts.

The compounds of the invention may be formulated with one or more conventional carriers and/or excipients according to techniques well known in the art. Typically, the compositions will be adapted for oral or parenteral administration, for example by intradermal, subcutaneous, intraperitoneal or intravenous injection.

For example, these may be formulated in conventional oral administration forms, e.g. tablets, coated tablets, capsules, powders, granulates, solutions, dispersions, suspensions, syrups, emulsions, etc. using conventional excipients, e.g. solvents, diluents, binders, sweeteners, aromas, pH modifiers, viscosity modifiers, antioxidants, etc. Suitable excipients may include, for example, corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose or fatty substances such as saturated fats or suitable mixtures thereof, etc.

Where parenteral administration is employed this may for example be by means of intravenous, subcutaneous or intramuscular injection. For this purpose, sterile solutions containing the active agent may be employed, such as an oil-in-water emulsion. Where water is present, an appropriate buffer system (e.g., sodium phosphate, sodium acetate or sodium borate) may be added to prevent pH drift under storage conditions.

The use of orally administrable compositions, e.g. tablets, coated tablets, capsules, syrups, etc. is especially preferred.

The formulations may be prepared using conventional techniques, such as dissolution and/or mixing procedures.

The dosage required to achieve the desired activity of the compounds herein described will depend on various factors, such as the compound selected, its mode and frequency of administration, whether the treatment is therapeutic or prophylactic, and the nature and severity of the disease or condition, etc. Typically, a physician will determine the actual dosage which will be most suitable for an individual subject. The specific dose level and frequency of dosage for any particular patient may be varied and will depend upon factors such as the activity of the specific compound employed, the metabolic stability and length of action of that compound, the age of the patient, the mode and time of administration, and the severity of the particular condition. The compound and/or the pharmaceutical composition may be administered in accordance with a regimen from 1 to 10 times per day, such as once or twice per day. For oral and parenteral administration to human patients, the daily dosage level of the agent may be in single or divided doses.

Suitable daily dosages of the compounds herein described are expected to be in the range from 0.1 mg to 1 g of the compound; 1 mg to 500 mg of the compound; 1 mg to 300 mg of the compound; 5 mg to 100 mg of the compound, or 10 mg to 50 mg of the compound. By a “daily dosage” is meant the dosage per 24 hours.

The invention will now be described in more detail in the following non-limiting Examples and with reference to the accompanying figures, in which:

FIG. 1 shows the protection of compound BG-1 against limited tryptic proteolysis of WT-HMBS. (A) SDS PAGE showing the effect of the indicated compound (84 μM and 2% DMSO) with HMBS. Std, low molecular weight standards; Control n.t., no trypsin added; Control DMSO, HMBS with 2% DMSO and trypsin; BG-1, HMBS with 2% DMSO, trypsin and compound BG-1. (B) Quantification of the lowest 31.5 kDa band relative to the full-length HMBS at 42.5 kDa. **p<0.01 for differences compared to the DMSO control, calculated by two-sample student's t-test for equal variance.

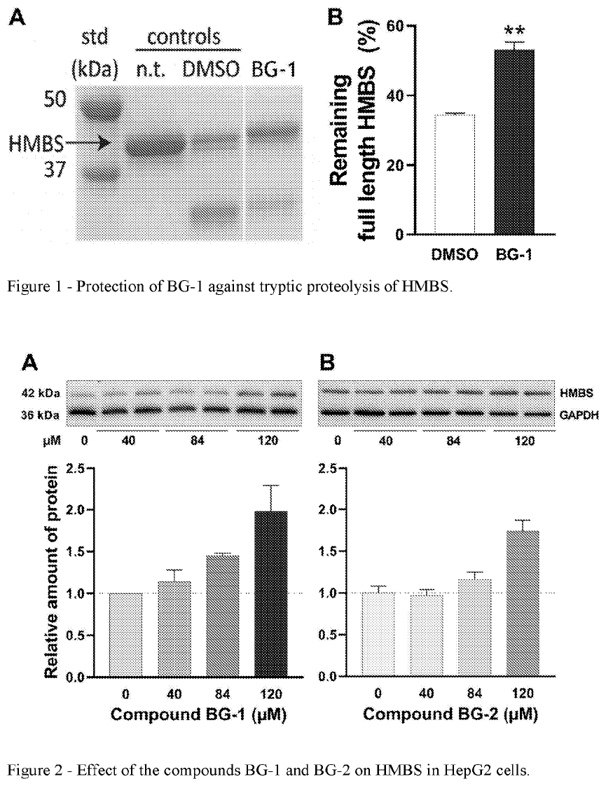

FIG. 2 shows western blotting and immunoquantification of the relative amount of HMBS in cell lysates from WT-HMBS stably transfected HepG2 cells treated with either compound BG-1 (A) or compound BG-2 (B) at the indicated concentrations. DMSO (2%) was included in all samples. Representative blots are shown, and the histograms below represent the quantification of the relative HMBS levels (n=3), using GAPDH as the protein loading control.

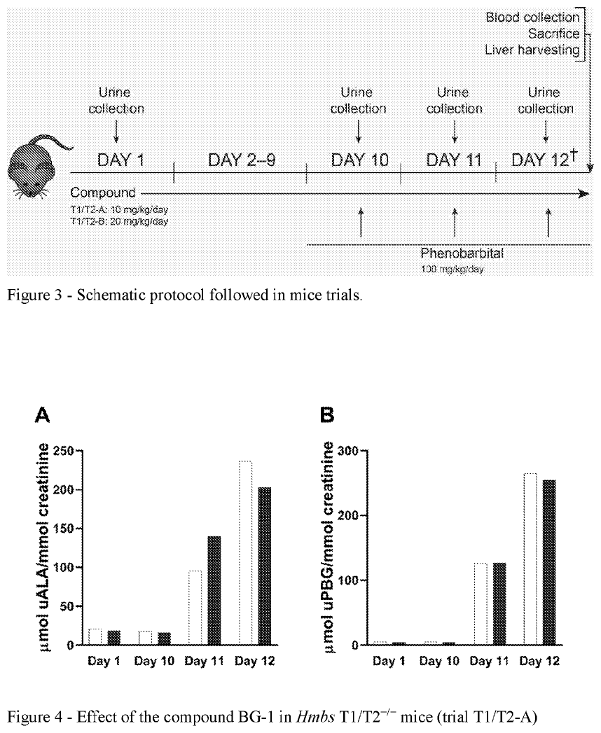

FIG. 3 shows the schematic protocol followed in the mice trials. Female compound heterozygote Hmbs-deficient T1/T2^{-/-} mice were kept on normal diet and given 10 or 20 mg/kg/day (trial T1/T2-A and T1/T2-B, respectively) of either the desired compound or DMSO, by oral gavage, for 12 consecutive days. Biochemical acute attack was induced by intraperitoneal injection of phenobarbital days 10-12. Urine was collected on day 1 and 10-12, and blood samples were collected before—and livers were harvested after—sacrifice.

FIG. 4 shows the effect of the compound BG-1 in Hmbs-deficient mice (trial T1/T2-A). One group of Hmbs T1/T2^{-/-} mice (n=6) were orally treated for 12 days with 10 mg/kg/day of compound BG-1. On day 10, 11 and 12 they were induced by phenobarbital. A control group was given 10% DMSO, and likewise induced with phenobarbital. On days 1 and 10-12 urine was collected and pooled from each group before measurement. (A,B) Bars represent porphyrin precursor ALA (A) and PBG (B) in urine from control group (white) and compound BG-1 (blue).

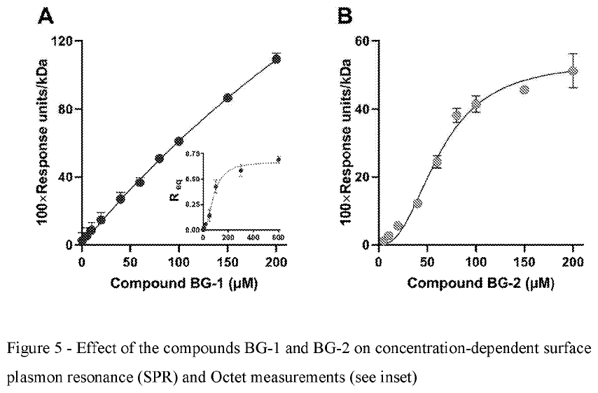

FIG. 5 shows concentration-dependent SPR with HMBS immobilized to sensor chip. (A) No apparent concentration for half-maximal binding (S_{0.5})-value was obtained for compound BG-1 with SPR. The data from the Octet measurements (A; inset) provided an S_{0.5}=83±7. (B) An S_{0.5}=63±3 μM was obtained, also using sigmoidal fitting, from the binding isotherm for compound BG-2.

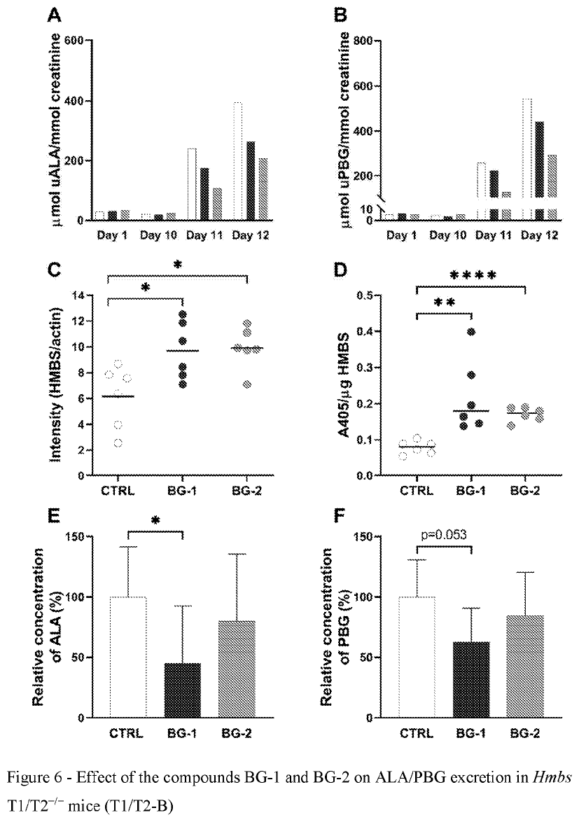

FIG. 6 shows the effect on ALA/PBG excretion of the compound BG-1 and compound BG-2 in Hmbs-deficient mice (trial T1/T2-B). Two groups of Hmbs T1/T2^{-/-} mice (n=6 in each group) were treated for 12 days with 20 mg/kg/day of either compound BG-1 or compound BG-2. I.p. injection of phenobarbital was given on days 10-12 to induce the heme biosynthesis, and thus precipitation of biochemical acute attack. A control group was given 10% DMSO and likewise induced with phenobarbital. Urine was collected on day 1 and 10-12, and livers were harvested after sacrifice. Protein levels were measured in liver lysates by western blot quantification. (A,B) Urine from the mice was pooled for each group and bars represent porphyrin precursors ALA (A) and PBG (B) treated with compound BG-1 (blue) and compound BG-2 (green). Control group is shown in white. (C) Scatter plots (circles) with mean (line) representing HMBS protein levels in mice livers treated with compound BG-1 (blue), and compound BG-2 (green). *p<0.05 for differences compared to the corresponding control (10% DMSO without compound; white circles), calculated by unpaired two-tailed t-test. (D) Scatter plot (circles) with mean (line) showing the enzymatic activity in liver tissue. **p<0.01 and ****p<0.0001 for differences compared to the corresponding control (10% DMSO without compound), calculated by unpaired two-tailed t-test. (E,F) The relative concentrations of ALA (E) and PBG (F) were measured in liver tissue extracts after treatment with compound BG-1 (blue) and compound BG-2 (green). *p<0.05 for differences compared to the corresponding control (10% DMSO without compound, white), calculated unpaired two-tailed t-test)

### EXAMPLES

### General Procedures

### Expression and Purification of HMBS Proteins:

WT-HMBS was expressed and purified to apparent homogeneity as previously described (Bustad et al., Bioscience reports 2013; 33(4)). The protein was further purified by size exclusion chromatography with a Superdex™ 75 10/300 GL column (GE Healthcare) in 20 mM HEPES, 150 mM NaCl, pH 8.2 and stored as aliquots in liquid N2 until use.

The enzyme used in the binding assays using the Octet RED96 was expressed using a new construct with an N-terminal 6×HIS affinity tag and a TEV protease cleavage site. Full-length HMBS was cloned into pET-28a(+)-TEV vector and transformed into BL21 (DE3) cells for expression. Expression was done in Terrific Broth medium with IPTG induction. Cells were cultured 16 h at 20° C. with 220 rpm shaking. After harvesting, the cells were lysed with sonication, and standard affinity purification was performed using Ni-NTA affinity matrix. Protein was eluted with 20 mM HEPES, 150 mM NaCl (gel filtration buffer), pH 8 supplemented with 400 mM imidazole. Affinity tag was cleaved overnight and removed with passing the protein through Ni-NTA. The protein was further purified by size exclusion chromatography with a Superdex™ 75 10/300 GL or 16/60 PG column (GE Healthcare, Chicago, Ill.) in gel filtration buffer, pH 8.0, and stored as aliquots in liquid N2 until use.

### Enzymatic Activity Assay of Recombinant HMBS:

The standard enzymatic activity of recombinant WT-HMBS was assayed at 37° C. as reported previously (Bustad et al., Bioscience reports, 2013; 33(4)). Compounds were added at a concentration of 84 μM. Absorbance of uroporphyrinogen I was determined at 405 nm. The enzyme activity in the presence of the compounds was normalized relative to DMSO-control (relative activity).

The effect of the compounds on the stability of HMBS activity was assayed by pre-incubating the HMBS (4-5 μg) in 50 mM HEPES pH 8.2, 84 μM compound and 2% DMSO for 20 min at 70° C., and then placed on ice for 5 min. The enzymatic activity at 37° C. was subsequently measured as reported previously (Bustad et al., Bioscience reports 2013; 3 3(4)). Controls without compound but with equal concentration of DMSO were included. The remaining activity in the presence of compounds was normalized relative to DMSO-control (relative activity). Kₘ and Vₘₐₓ were determined using an increasing concentration of PBG (3.125-1000 μM), and the kinetic parameters were obtained by non-linear curve fitting to Michaelis-Menten enzyme kinetics using GraphPad Prism version 8.2.0 for Windows, GraphPad Software, La Jolla Calif. USA, www.graphpad.com.

### Limited Proteolysis by Trypsin:

Limited proteolysis by trypsin was performed at 37° C. in 20 mM HEPES, 150 mM NaCl, 2% DMSO, pH 8.2, with 0.15 μg/μl HMBS in the absence (DMSO-control) or presence of 84 μM compound and 2% DMSO. The proteolysis was initiated by adding 1 μg/ml TPCK-treated trypsin (Sigma-Aldrich). After 30 min, aliquots were removed and transferred to Laemmli loading buffer containing 2 μg/ml soybean trypsin inhibitor. Samples resolved by electrophoresis with 10% Mini-Protean® TGX™ gels (Bio-Rad Laboratories, Inc.) were analyzed using the Image Lab™ software (Bio-Rad Laboratories, Inc.). The unpaired t-test (two tailed) was performed using GraphPad Prism.

### Transfection of HepG2 Cells and Growth in the Presence of Compounds:

The human hepatoma HepG2 cells were obtained from Leibniz-Institut DSMZ—Deutsche Sammlung von Mikroorganismen und Zellkulturen GmbH. Cells were maintained in RPMI 1640, GlutaMAX™ (Thermo Fisher Scientific) medium supplemented with 10% heat-inactivated fetal bovine serum and 1% penicillin-streptomycin (Thermo Fisher Scientific) in a humidified incubator with 5% CO₂ at 37° C. HMBS cDNA was inserted into the pcDNA3.1(+) cloning vector (Thermo Fisher Scientific). The HepG2 cells were then transfected with the pcDNA3.1(+) vector containing HMBS using FuGENE®HD Transfection Reagent (Promega, Madison, Wis.) according to the manufacturer's recommendations. Stably transfected clones were selected for resistance to the neomycin analogue G418 (Thermo Fisher Scientific). WT-HMBS transfected HepG2 cells (2×10⁶) were seeded and grown for 22 h before compounds were added to final concentrations of 0, 40, 84, 120 and 168 μM in 2% DMSO. Cells were harvested after 24 h and analyzed as described below.

### Surface Plasmon Resonance (SPR):

Surface plasmon resonance experiments for the estimation of the concentration of compound at half-maximal binding (S_{0.5}) were performed using a Biacore T200 (GE Healthcare) instrument at 25° C. 150 μg/ml WT-HMBS in 10 mM sodium acetate pH 4.5 was immobilized onto a CM5-S sensor chip through amine-coupling chemistry and PBS containing 0.05% surfactant P20 as running buffer, reaching immobilization levels ˜15,000. The baseline was equilibrated for 1-2 h, before the compounds were assayed in a concentration-dependent manner (0-200 μM), using running buffer with 5% DMSO and 30 μl/min flow rate. Contact and dissociation time was 60 s, with a final wash after 50% DMSO injection. Blank immobilization, solvent correction and negative control (assay buffer) were included for the analysis of the sensorgrams using the Biacore T200 Evaluation software v2.0. The allosteric sigmoidal curve fitting was performed using GraphPad Prism.

### Octet RED96:

Octet RED96 system (ForteBio Biologics by Molecular Devices, LLC., San Jose, Calif.) with super streptavidin (SSA) biosensors was used as an additional method for determining the K_{d}-value for the binding of the compounds. Loading HMBS to the SSA sensors required biotinylation, which was carried out at room temperature mixing 1.5 molar excess of NHS-ester biotinylation reagent (EZ-Link™ NHS-PEG4-Biotin, Thermo Fisher Scientific) to protein. After 30 minutes excess of biotin was removed using Zeba spin desalting column (Thermo Fisher Scientific) and a gel filtration buffer was changed to reaction buffer, PBS-P+(GE Healthcare) supplemented with 5% DMSO. Sensors were loaded with 5 μg/ml of biotinylated HMBS, reaching 6 nm surface thickness. Triplicates for the concentration series of the compounds were measured, and double reference subtraction was applied for data analysis based on the steady-state kinetics with equilibrium binding signal (Req) using ForteBio Data analysis 9.0. The allosteric sigmoidal curve fitting was performed using GraphPad Prism.

### Animal Studies:

The compound heterozygote Hmbs-deficient T1/T2^{-/-} mouse model (Lindberg et al., Nature genetics, 1996; 12(2):195-9) was utilized in the two sets of animal studies performed, T1/T2-A and T1/T2-B: i) In the T1/T2-A study mice (2-4 months old, 16-22 g) were given 10 mg/kg/day of compound BG-1. ii) In the T1/T2-B study, mice (2-3 months old, 17-22 g) were treated with 20 mg/kg/day of either compound BG-1 or compound BG-2. The mice in both groups were given phenobarbital (Gardenal®) at 100 mg/kg through i.p. injection on days 10-12 of the study. Urine from these mice was collected day 1 before start of treatment, and each day of phenobarbital injection (day 10, 11 and 12). The protocol is presented in FIG. 3.

Compounds were dissolved in 10% DMSO and all studies included treatment groups with 6 mice in each, including a control group given only 10% DMSO. The compounds or DMSO alone were administered for twelve consecutive days and the mice were sacrificed 30 min after the last dose of compound or phenobarbital. The mice were anaesthetized by i.p. injection of tribromoethanol (3 mg/kg), blood samples collected on EDTA by retro-orbital puncture and livers harvested and flash frozen in liquid N2 before storage at −80° C.

Pooled urinary porphyrin precursor levels (ALA and PBG) were analyzed by sequential ion-exchange chromatography using the ALA/PBG by Column Test (Bio-Rad Laboratories, Inc.) according to the manufacturer's recommendations.

### Cell and Tissue Sampling:

HepG2 cells were washed in ice-cold PBS before 10 min lysis on ice with cold RIPA buffer (25 mM Tris-HCl, 150 mM NaCl, 1% NP-40, 1% sodium deoxycholate, 0.1% SDS (Cell Signaling Technologies, Danvers, Mass.) and cOmplete protease inhibitor cocktail (Roche Diagnostics)). Lysates were centrifuged (10,000 g, 15 min) and supernatants were removed and stored at −80° C. until further use. Frozen liver tissue was homogenized in 50 mM Tris-HCl, pH 7.4, 100 mM KCl, 1 mM DTT, 0.2 mM PMSF, 1 mM benzamidine, 1 mM EDTA and 1 tablet/10 ml cOmplete ULTRA protease inhibitor cocktail (Roche Diagnostics) using TissueLyser II (Qiagen, Venlo, Netherlands). The extracts were clarified by centrifugation at 14,000 g for 20 min at 4° C., and supernatants were stored at −80° C.

### Enzymatic Activity Assay of HMBS in Liver Tissue:

The crude liver homogenates were passed through Zeba spin desalting columns (Thermo Fisher Scientific) to remove small molecules <2000 Da. 50 mM HEPES pH 8.2 was used as equilibration buffer. 25 μl filtrated homogenate (400-500 μg total protein) was added to 110 μl sample mix (50 mM HEPES, pH 8.2, 1% Triton™ X-100) and incubated for 10 min at 37° C. before adding PBG (1 mM). The reaction was stopped after 1 h by adding ice-cooled 100% TCA to a final concentration of 25%, incubated at room temperature (RT) for 10 min and centrifuged at 10,000 g for 10 min. The absorption was measured in the supernatant at A409 with baseline correction at A380 using NanoDrop™ 2000c spectrophotometer (Thermo Fisher Scientific). The activity of HMBS was expressed as relative activity in the compound-treated cell compared to DMSO-controls. A blank sample was prepared for each homogenate. The unpaired t-test (two tailed) was performed using GraphPad Prism.

### Quantitative Detection of HMBS in Cell Lysate and Tissue by Immunoblot:

Cell lysate samples (20 μg total protein) were separated by electrophoresis and subsequently transferred to a PVDF transfer membrane (Bio-Rad). Membranes were blocked for 1 hour at RT with 5% non-fat dry milk (Bio-Rad Laboratories) in Tris-buffered saline (TBS; 20 mm Tris-HCl, 140 mM NaCl pH 7.4), containing 0.1% Tween® (0.1% TBS-T). Immunoblotting was carried out with 1:1,000 anti-HMBS primary Ab (H300; Santa Cruz Biotechnology, Dallas, Tex.) in 0.1% TBS-T, overnight at 4° C. Subsequently the membranes were washed extensively in 0.1% TBS-T followed by 1 h incubation at RT with HRP-conjugated goat-anti-rabbit IgG secondary Ab (Bio-Rad) 1:5,000 dilution. Anti-GAPDH (Abcam, Cambridge, UK) was used as loading control. Chemiluminescence of secondary Ab-HRP conjugates was elicited using Luminata™ Crescendo Western HRP Substrate (Merck Millipore, Burlington, Mass.), imaged with Gel Doc™ XR+ (Bio-Rad) and quantified using Image Lab software (Bio-Rad).

Liver homogenates (5 μg total protein/lane) were loaded onto 10% Mini-Protean® TGX™ gels (Bio-Rad) and separated with Tris/Glycine/SDS electrophoresis buffer (Bio-Rad). Trans-Blot® Turbo™ Transfer Starter System (Bio-Rad) was used to transfer the proteins onto Immun-Blot® low fluorescence PVDF membranes (Bio-Rad). The membranes were then blocked with TBS containing 1% Tween® 20 (1% TBS-T) and 3% BSA for 1 h. HMBS was probed with 1:2,000 monoclonal mouse anti-HMBS (H-11, Santa Cruz Biotechnology), together with 1:1,000 rabbit anti-actin (Sigma-Aldrich), in 1% TBS-T, 3% BSA overnight, 4° C. Alexa Fluor 647 conjugated donkey-anti-mouse and Alexa Fluor 488 conjugated donkey-anti-rabbit (both Thermo Fisher Scientific) were used as secondary antibodies and incubated in 1:1,000 dilutions in 0.1% TBS-T for 1 h. Each step was followed by extensive wash with 0.1% TBS-T. Fluorescence detection was performed using G-Box Chemi-XRQ (Syngene Synoptics, Cambridge, UK) with filters UV06 and 705 nm for AF-488 and AF-647, respectively, and the band intensities of HMBS relative to the loading control (actin) were determined using ImageJ (Schneider et al., Nature methods 2012; 9(7): 671-5). Plotting and the two-tailed unpaired t-test were performed using GraphPad Prism version 8.2.0.

### Determination of Compound and Metabolites in Liver Tissue Samples:

One volume of homogenized liver tissue was mixed with two volumes of acetonitrile:MetOH (1:1, v/v) and centrifuged. High performance liquid chromatography/tandem mass spectrometry (HPLC-MS/MS) was used to determine the concentration of compound BG-1, ALA and PBG in the supernatants. Analyses of samples were conducted by the Bioanalytical Laboratory personnel at Enamine/Bienta (Bienta/Enamine Ltd Biology Services, Kiev, Ukraine, www.bienta.net). The unpaired t-test (one tailed) was performed using GraphPad Prism version 8.2.0.

### Statistics:

Results are presented as mean±SD, except for relative data where relative mean is presented with error of propagation. Statistical comparisons were done using two-sample student's t-test for equal variance, and statistically significance was defined as p<0.05 or lower, as specified in the text. All statistical analyses and plotting of data were performed in GraphPad Prism 8.2.0.

NMR spectroscopy:

¹H NMR spectra were recorded at 400 MHz on a Bruker Avance III NMR spectrometer. Samples were prepared in deuterated chloroform (CDCl₃) or dimethylsulphoxide (DMSO-d₆) and the raw data were processed using the ACD NMR software.

### UPLC-MS Analysis:

LCMS analysis was conducted on a Waters Acquity UPLC system consisting of an Acquity i-Class Sample Manager-FL, Acquity i-Class Binary Solvent Manager and Acquity i-Class UPLC Column Manager. UV detection was achieved using an Acquity i-Class UPLC PDA detector (scanning from 210 to 400 nm), whereas mass detection was achieved using an Acquity QDa detector (mass scanning from 100-1250 Da; positive and negative modes simultaneously). A Waters Acquity UPLC BEH C18 column (2.1×50 mm, 1.7 μm) was used to achieve the separation of the analytes.

Samples were prepared by dissolving (with or without sonication) into 1 ml of a 1:1 (v/v) mixture of MeCN in H₂O. The resulting solutions were filtered through a 0.2 μm syringe filter before being submitted for analysis. All of the solvents (including formic acid and 36% ammonia solution) used were used as the HPLC grade. Four different analytical methods were used, the details of which are presented below.

Acidic run (2 min): 0.1% v/v Formic acid in water [Eluent A]; 0.1% v/v Formic acid in MeCN [Eluent B]; Flow rate 0.8 ml/min; injection volume 2 μl and 1.5 min equilibration time between samples.

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

Acidic run (4 min): 0.1% v/v formic acid in water [Eluent A]; 0.1% v/v formic acid in MeCN [Eluent B]; Flow rate 0.8 ml/min; injection volume 2 μl and 1.5 min equilibration time between samples.

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

Basic run (2 min): 0.1% ammonia in water [Eluent A]; 0.1% ammonia in MeCN [Eluent B]; Flow rate 0.8 ml/min; injection volume 2 μl and 1.5 min equilibration time between samples.

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

Basic run (4 min): 0.1% ammonia in water [Eluent A]; 0.1% ammonia in MeCN [Eluent B]; Flow rate 0.8 ml/min; injection volume 2 μl and 1.5 min equilibration time between samples.

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

### Example 1—Synthesis of (4-chloro-3-(trifluoromethyl)phenyl)(phenyl)methanone

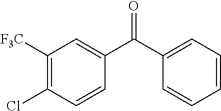 [SMILES: O=C(c1ccccc1)c1ccc(Cl)c(C(F)(F)F)c1]

To a stirred solution of 4-bromo-1-chloro-2-(trifluoromethyl)benzene (314 mg, 1.21 mmol) in THF (6.0 ml) at −78° C. was added a solution of n-BuLi in hexanes (1.6 M, 0.58 ml, 1.45 mmol), and the resulting mixture was stirred at −78° C. for 30 min. A solution of N-methoxy-N-methylbenzamide (200 mg, 1.21 mmol) in THF (0.5 ml) was added to the reaction, and the resulting mixture was allowed to warm to room temperature with stirring over 60 min. The mixture was quenched by the addition of a saturated aqueous solution of ammonium chloride (10 ml) and extracted with DCM (2×10 ml). The combined organics were dried over sodium sulphate, filtered and evaporated to dryness to give a residue. Purification by flash column chromatography over silica gel (Biotage) eluting with a gradient of EtOAc (0 to 10%) in hexanes gave the desired product as a white solid (64 mg, Y=18%).

UPLC-MS (Acidic Method, 4 min): rt=2.22 min, m z=n.d. [M+H]⁺

¹H NMR (400 MHz, Chloroform-d) δ 8.15 (d, J=2.0 Hz, 1H), 7.91 (dd, J=8.3, 2.1 Hz, 1H), 7.81-7.74 (m, 2H), 7.69-7.61 (m, 2H), 7.57-7.48 (m, 2H).

### Example 2—Synthesis of (6-chloropyridin-3-yl)(phenyl)methanone

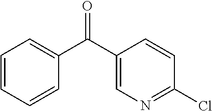 [SMILES: O=C(c1ccccc1)c1ccc(Cl)nc1]

This compound was prepared according to the standard procedure for the formation of ketones via the addition of an organolithium species to a Weinreb amide in Example 1, using 5-bromo-2-chloropyridine (150 mg, 0.91 mmol) and N-methoxy-N-methylbenzamide (175 mg, 0.91 mmol). The desired product was isolated as a green oil (71.3 mg, Y=36%)

UPLC-MS (Acidic Method, 4 min): rt=1.67 min, m z=218 [M+H]⁺

¹H NMR (400 MHz, Chloroform-d) δ 8.77 (d, J=2.4, 0.8 Hz, 1H), 8.10 (dd, J=8.3, 2.4 Hz, 1H), 7.84-7.77 (m, 2H), 7.68-7.62 (m, 1H), 7.56-7.50 (m, 2H), 7.50-7.46 (m, 1H).

### Example 3—Synthesis of (4-chloro-3-nitrophenyl)(phenyl)methanol

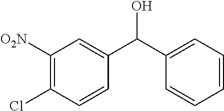 [SMILES: O=[N+]([O-])c1cc(C(O)c2ccccc2)ccc1Cl]

Sodium borohydride (72.3 mg, 1.91 mmol) was added in a single portion to a stirred solution of (4-chloro-3-nitrophenyl)(phenyl)methanone (200 mg, 0.76 mmol) in methanol (5 ml) at 0° C., and the resulting mixture was warmed to room temperature with stirring for 16 h. The mixture was quenched by the addition of a saturated aqueous solution of ammonium chloride (10 ml) and evaporated to dryness to give a residue. Purification by flash column chromatography over silica gel (Biotage) eluting with a gradient of EtOAc (0 to 50%) in hexanes gave the desired product as a white solid (105 mg, Y=52%).

UPLC-MS (Acidic Method, 4 min): rt=1.80 min, m z=n.d. [M+H]⁺

¹H NMR (400 MHz, Chloroform-d) δ 7.95 (d, J=1.8 Hz, 1H), 7.53-7.46 (m, 2H), 7.41-7.29 (m, 5H), 5.86 (d, J=3.1 Hz, 1H), 2.39 (d, J=3.2 Hz, 1H).

### Example 4—Synthesis of 4-benzyl-1-chloro-2-nitrobenzene

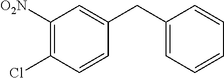 [SMILES: O=[N+]([O-])c1cc(Cc2ccccc2)ccc1Cl]

A stirred solution of (4-chloro-3-nitrophenyl)(phenyl)methanone (250 mg, 0.95 mmol), triethylsilane (0.99 ml, 6.2 mmol) and boron trifluoride diethyl etherate (0.79 ml, 6.42 mmol) was heated at 65° C. for 2 h. The mixture was quenched by the addition of a saturated aqueous solution of sodium bicarbonate (10 ml) and extracted with EtOAc (3×10 ml). The combined organics were dried over sodium sulphate, filtered and evaporated to dryness to give a residue. Purification by flash column chromatography over silica gel (Biotage) eluting with a gradient of EtOAc (0 to 30%) in hexanes gave the desired product as a white solid (209 mg, Y=89%).

UPLC-MS (Acidic Method, 4 min): rt=2.14 min, m z=n.d. [M+H]⁺

¹H NMR (400 MHz, Chloroform-d) δ 7.67 (d, J=2.1 Hz, 1H), 7.43 (d, J=8.2 Hz, 1H), 7.35-7.29 (m, 3H), 7.27-7.22 (m, 1H), 7.19-7.13 (m, 2H), 4.01 (s, 2H).

### Example 5—Synthesis of 4-chloro-N-methyl-3-nitro-N-phenylbenzamide

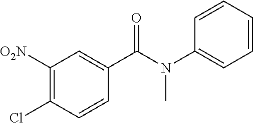 [SMILES: CN(C(=O)c1ccc(Cl)c([N+](=O)[O-])c1)c1ccccc1]

A stirred solution of 4-chloro-3-nitro-N-phenylbenzamide (222 mg, 0.8 mmol) in DMF (2 Ml) was treated with sodium hydride (60% dispersion, 39 mg, 0.96 mmol), and the resulting mixture was stirred for 30 min. Iodomethane (60 ml, 0.96 mmol) was added to the reaction, and the resulting mixture was stirred for 16 h. The reaction was quenched by the addition of a saturated aqueous solution of ammonium chloride (20 ml) and extracted with EtOAc (3×20 ml). The combined organics were dried over sodium sulphate, filtered and evaporated to dryness to give a residue. Purification by flash column chromatography over silica gel (Biotage) eluting with a gradient of EtOAc (0 to 50%) in hexanes gave the desired product as a white solid (74 mg, Y=32%).

UPLC-MS (Acidic Method, 4 min): rt=1.79 min, m z=277 [M+H]⁺

¹H NMR (400 MHz, DMSO-d6) δ 7.96 (s, 1H), 7.63 (d, J=8.4 Hz, 1H), 7.50 (d, J=8.4 Hz, 1H), 7.36-7.18 (m, 6H), 3.38 (s, 3H).

### Example 6—Synthesis of (4-chloro-3-nitrophenyl)(2,6-dimethylphenyl)methanone

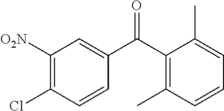 [SMILES: Cc1cccc(C)c1C(=O)c1ccc(Cl)c([N+](=O)[O-])c1]

Activated manganese(IV) oxide (178 mg, 2.05 mmol) was added to a solution of (4-chloro-3-nitrophenyl)(2,6-dimethylphenyl)methanol (120 mg, 0.41 mmol) in dichloromethane (10 mL) at ambient temperature, and the resulting mixture was stirred at ambient temperature for 16 h. The reaction mixture was filtered through a plug of Celite, washing with DCM. The combined filtrate was evaporated to dryness to give the crude product as a residue. Purification by flash column chromatography over silica gel (Biotage) eluting with a gradient of EtOAc (0 to 5%) in hexanes gave the desired product as a white solid (45 mg, Y=38%).

UPLC-MS (Acidic Method, 2 min): rt=1.28 min, m z=n.d. [M+H]⁺

¹H NMR (400 MHz, Chloroform-d) δ 8.24 (d, J=2.0 Hz, 1H), 7.90 (dd, J=8.4, 2.0 Hz, 1H), 7.66 (d, J=8.4 Hz, 1H), 7.30 (t, J=7.7 Hz, 1H), 7.12 (d, J=7.6 Hz, 2H), 2.12 (s, 6H).

### Example 7—Synthesis of (4-chloro-3-nitrophenyl)(4-(trifluoromethoxy)phenyl)methanone

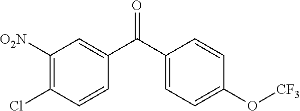 [SMILES: O=C(c1ccc(OC(F)(F)F)cc1)c1ccc(Cl)c([N+](=O)[O-])c1]

Activated manganese(IV) oxide (125 mg, 149 mmol) was added to a solution of (4-chloro-3-nitrophenyl)(4-(trifluoromethoxy)phenyl)methanol (100 mg, 0.29 mmol) in dichloromethane (10 ml) at ambient temperature, and the resulting mixture was stirred at ambient temperature for 16 h. The reaction mixture was filtered through a plug of Celite, washing with DCM. The combined filtrate was evaporated to dryness to give the crude product as a residue. Purification by flash column chromatography over silica gel (Biotage) eluting with a gradient of EtOAc (0 to 30%) in hexanes gave the desired product as a white solid (60 mg, Y=60%).

UPLC-MS (Acidic Method, 4 min): rt=2.20 min, m z=n.d. [M+H]⁺

¹H NMR (400 MHz, Chloroform-d) δ 8.27 (d, J=2.0 Hz, 1H), 7.94 (dd, 1H), 7.89-7.82 (m, 2H), 7.72 (d, J=8.3 Hz, 1H), 7.39-7.35 (m, 2H).

### Example 8—Synthesis of (4-chloro-3-nitrophenyl)(4-(trifluoromethoxy)phenyl)methanol

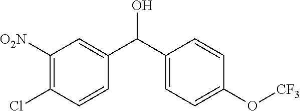 [SMILES: O=[N+]([O-])c1cc(C(O)c2ccc(OC(F)(F)F)cc2)ccc1Cl]

A solution of (4-(trifluoromethoxy)phenyl)magnesium bromide in THF (0.5 M, 11.0 ml, 5.5 mmol) was added dropwise to a stirred solution of 4-chloro-3-nitrobenzaldehyde (1.0 g, 5.39 mmol) in anhydrous THF (40 ml) at −78° C., and the resulting mixture was allowed to warm to ambient temperature with stirring for 16 h. The mixture was quenched by the addition of a saturated aqueous solution of ammonium chloride (10 ml) and extracted with EtOAc (2×10 ml). The combined organics were dried over sodium sulphate, filtered and evaporated to dryness to give a residue. Purification by flash column chromatography over silica gel (Biotage) eluting with a gradient of EtOAc (0 to 30%) in hexanes gave the desired product as a pale yellow oil (1.1 g, 69%).

UPLC-MS (Acidic Method, 2 min): rt=1.22 min, m z=346 [M−H]⁺

¹H NMR (400 MHz, Chloroform-d) δ 7.94 (d, J=1.9 Hz, 1H), 7.55-7.45 (m, 2H), 7.39 (d, J=8.6 Hz, 2H), 7.25-7.18 (m, 2H), 5.88 (s, 1H), 2.41 (s, 1H).

### Example 9—Synthesis of (4-chloro-3-(trifluoromethyl)phenyl)(4-(trifluoromethoxy) phenyl)methanone

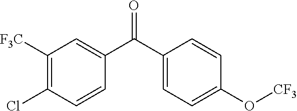 [SMILES: O=C(c1ccc(OC(F)(F)F)cc1)c1ccc(Cl)c(C(F)(F)F)c1]

Activated manganese(IV) oxide (469 mg, 5.4 mmol) was added to a solution of (4-chloro-3-(trifluoromethyl)phenyl)(4-(trifluoromethoxy)phenyl)methanol (400 mg, 1.08 mmol) in dichloromethane (6.2 mL) at ambient temperature, and the resulting mixture was stirred at ambient temperature for 16 h. The reaction mixture was filtered through a plug of Celite, washing with DCM. The combined filtrate was evaporated to dryness to give the crude product as a residue. Purification by flash column chromatography over silica gel (Biotage) eluting with a gradient of EtOAc (0 to 20%) in hexanes gave the desired product as a white solid (93 mg, Y=24%).

UPLC (4 min, acidic): rt=2.46 min, no mass ionisation, 100% purity by UV.

¹H NMR (400 MHz, Chloroform-d) δ 8.13 (d, J=2.1 Hz, 1H), 7.92-7.80 (m, 3H), 7.66 (d, J=8.3 Hz, 1H), 7.40-7.31 (m, 2H).

19F NMR (DMSO-d6) δ: −62.33, −67.60

### Example 10—Synthesis of (4-chloro-3-nitrophenyl)(4-methoxyphenyl)methanol

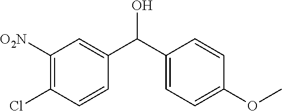 [SMILES: COc1ccc(C(O)c2ccc(Cl)c([N+](=O)[O-])c2)cc1]

A solution of 4-methoxyphenyl)magnesium in THF (0.5 M, 11.0 ml, 5.5 mmol) was added dropwise to a stirred solution of 4-chloro-3-nitrobenzaldehyde (1.0 g, 5.39 mmol) in anhydrous THF (40 mL) at −78° C., and the resulting mixture was allowed to warm to ambient temperature with stirring for 16 h. The mixture was quenched by the addition of a saturated aqueous solution of ammonium chloride (10 ml) and extracted with EtOAc (2×10 ml). The combined organics were dried over sodium sulphate, filtered and evaporated to dryness to give a residue. Purification by flash column chromatography over silica gel (Biotage) eluting with a gradient of EtOAc (0 to 30%) in hexanes gave the desired product as a pale yellow oil (1.1 g, 67%).

UPLC (4 min, acidic): rt=1.81 min, no mass ionisation, 100% purity by UV.

¹H NMR (DMSO-d6) δ: 8.03 (d, J=1.9 Hz, 1H), 7.70 (d, J=8.4 Hz, 1H), 7.63 (ddd, J=8.4, 2.1, 0.6 Hz, 1H), 7.33-7.25 (m, 2H), 6.92-6.84 (m, 2H), 6.16 (d, J=3.5 Hz, 1H), 5.77 (d, J=3.1 Hz, 1H), 3.72 (s, 3H).

### Example 11—Synthesis of 5-(2-chlorobenzyl)-2-hydroxybenzaldehyde

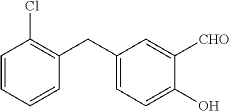 [SMILES: O=Cc1cc(Cc2ccccc2Cl)ccc1O]

A solution of 2-chlorobenzylzinc chloride in THF (0.5 M, 6.5 mL, 3.23 mmol) was added dropwise to a stirred suspension of 5-bromo-2-hydroxybenzaldehyde (500 mg, 2.49 mmol), SPhos (20 mg, 0.05 mmol), palladium(II) acetate (5.6 mg, 0.025 mmol) in anhydrous THF (2.5 mL) at 0° C., and the resulting mixture was allowed to warm to ambient temperature while stirring for 16 h. The mixture was quenched by the addition of a saturated aqueous solution of ammonium chloride (10 ml) and extracted with EtOAc (2×20 ml). The combined organics were dried over sodium sulphate, filtered and evaporated to dryness to give a residue. Purification by flash column chromatography over silica gel (Biotage) eluting with a gradient of EtOAc (0 to 10%) in hexanes gave the desired product as a colourless oil (250 mg, 41%).

UPLC (4 min, acidic): rt=2.08 min, no mass ionisation, 100% purity by UV.

¹H NMR (Chloroform-d) δ: 10.89 (s, 1H), 9.81 (s, 1H), 7.42-7.33 (m, 2H), 7.32 (d, J=2.3 Hz, 1H), 7.25-7.12 (m, 3H), 6.92 (d, J=8.5 Hz, 1H), 4.07 (s, 2H).

### Example 12—Synthesis of (3-nitro-4-(pyrrolidin-1-yl)phenyl)(4-(2-(pyrrolidin-1-yl) ethoxy)phenyl)methanone

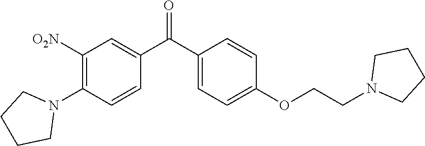 [SMILES: O=C(c1ccc(OCCN2CCCC2)cc1)c1ccc(N2CCCC2)c([N+](=O)[O-])c1]

To a solution of 4-(2-bromoethoxy)phenyl)(4-chloro-3-nitrophenyl)methanone (60 mg, 0.16 mmol) in anhydrous DMF (4.0 ml) was added K₂CO₃ (320 mesh, 86 mg, 0.62 mmol) and pyrrolidine (0.026 ml, 0.31 mmol), and the resulting suspension was stirred at 85° C. for 16 h. The reaction mixture was cooled to ambient temperature and partitioned between EtOAc (10 ml) and water (10 ml). The organic phase was separated and the aqueous phase washed with EtOAc (10 ml). The combined organic phases were dried over sodium sulphate, filtered and evaporated to dryness to give the crude product as an oil. Purification by flash column chromatography over silica gel (Biotage) eluting with a gradient of methanol (0 to 20%) in DCM gave the desired product as a solid (55 mg, Y=86%).

UPLC (4 min, acidic): rt=1.49 min. ESI(+)=410.3, 100% purity by UV

¹H NMR (Chloroform-d) δ: 8.13 (d, J=2.2 Hz, 1H), 7.84 (dd, J=9.0, 2.2 Hz, 1H), 7.72-7.64 (m, 2H), 6.96-6.88 (m, 2H), 6.87 (d, J=9.0 Hz, 1H), 4.24 (t, J=5.6 Hz, 2H), 3.29-3.21 (m, 4H), 3.02 (t, J=5.6 Hz, 2H), 2.78 (s, 4H), 2.02-1.91 (m, 4H), 1.89-1.81 (m, 4H).

### Example 13—Synthesis of 5-[(2-chlorophenyl)methyl]-2-hydroxy-3-(trifluoromethyl) benzaldehyde

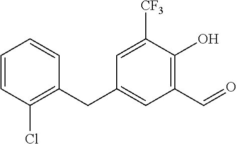 [SMILES: O=Cc1cc(Cc2ccccc2Cl)cc(C(F)(F)F)c1O]

To an oven-dried two-neck round bottom flask under a nitrogen atmosphere was added 5-bromo-2-hydroxy-3-(trifluoromethyl)benzaldehyde (500 mg, 1.86 mmol, 1.0 eq.), SPhos (15.3 mg, 0.037 mmol, 0.02 eq.), palladium(II) acetate (4.2 mg, 0.019 mmol, 0.01 eq.) and anhydrous THF (5.0 mL). The resulting orange solution was cooled to 0° C. before the dropwise addition of 2-chlorobenzyl zinc chloride (0.5 M in THF, 4.8 mL, 2.42 mmol, 1.3 eq.). The reaction was allowed to warm slowly to room temperature and stirred for 18 h. The reaction mixture was re-cooled to 0° C., quenched with saturated aqueous ammonium chloride solution (20 mL) and extracted with EtOAc (2×50 mL). The organic phase was separated, dried over sodium sulfate, filtered and concentrated in vacuo. The crude material was dry loaded on silica gel and purified by flash column chromatography (Biotage, 25 g Si, gradient elution 0-10% EtOAc/hexane) to afford the title compound (173 mg, 93% purity by UPLC) as a pale yellow semi-solid. A portion (50 mg) of this material was taken directly into the next step (see Example 14) and the remainder (120 mg) was further purified by prep-HPLC (C18, gradient 0-95% MeCN/H₂O+NH₄OH) and freeze dried to afford the title compound (38.0 mg, 0.12 mmol, 7%) as an off-white powder.

120 mg of the material was further purified by prep-HPLC (C18, gradient 0-95% MeCN/H₂O+NH₄OH) and freeze dried to afford the title compound (38.0 mg, 0.12 mmol, 7%) as an off-white powder.

UPLC MS (Acidic Method, 4 min): rt 2.29 min, ES⁻ m/z 313.1 [M-1]−, >99% purity by UV.

¹H NMR (400 MHz, DMSO) δ 10.07 (s, 1H), 11.48 (s, 1H), 7.80 (dd, J=17.1, 2.3 Hz, 2H), 7.46 (dd, J=7.5, 1.8 Hz, 1H), 7.42-7.37 (m, 1H), 7.31 (dtd, J=14.9, 7.4, 1.8 Hz, 2H), 4.14 (s, 2H). 97% purity.

¹⁹F NMR (376 MHz, DMSO) δ −60.99.

### Example 14—Synthesis of 4-(2-chlorobenzyl)-2-(hydroxymethyl)-6-(trifluoromethyl) phenol

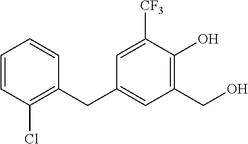 [SMILES: OCc1cc(Cc2ccccc2Cl)cc(C(F)(F)F)c1O]

To a cooled (0° C.) solution of 5-[(2-chlorophenyl)methyl]−2-hydroxy-3-(trifluoromethyl) benzaldehyde prepared in accordance with Example 13 (50.0 mg, 0.16 mmol, 1.0 eq.) in methanol (5.0 mL) was added sodium borohydride (12.0 mg, 0.32 mmol, 2.0 eq.) and the reaction was stirred at room temperature for 3 h. The reaction was concentrated in vacuo and the residue was dissolved in EtOAc (30 mL) and washed sequentially with water (30 mL) and brine (30 mL). The organic layer was dried over sodium sulfate, filtered and concentrated in vacuo. The resultant residue was purified by prep-HPLC (C18, gradient 0-95% MeCN/H₂O+NH₄OH) and freeze dried to afford the title compound (11.0 mg, 0.04 mmol, 22%) as an off-white powder.

UPLC MS (Acidic Method, 4 min): rt 2.04 min, ES⁻ m/z 315.1 [M-1]−, >99% purity by UV.

¹H NMR (400 MHz, DMSO) δ 7.44 (dd, J=7.6, 1.6 Hz, 1H), 7.37-7.22 (m, 5H), 4.57 (s, 2H), 4.03 (s, 2H).

¹⁹F NMR (376 MHz, DMSO) δ −60.29.

### Example 15—Activity and Proteolysis Assay

An enzymatic assay was performed to investigate the effect of compound BG-1 on the activity and conformational stability of HMBS. HMBS activity of the purified enzyme was measured in the absence and presence of compound BG-1 (at 84 μM) at standard conditions (37° C.) but also after pre-incubation at 70° C. for 20 min based on the high thermal stability of WT-HMBS. The results are presented in Table 1 below.

Limited tryptic proteolysis was also applied to compound BG-1. Proteolysis of WT-HMBS provided three major bands with relative content 34.5±0.3%, 14.7±0.8% and 50.9±0.5%, corresponding to remaining full-length HMBS (˜42.5 kDa), and two fragments of ˜41.0 kDa and ˜31.5 kDa, respectively (FIG. 1). Compound BG-1 exhibited protection against proteolysis (see FIG. 1). The effect of this compound at 84 μM on the steady-state enzyme kinetics of HMBS was then calculated (Table 2). The results showed a reduction in both K_{M} and Vₘₐₓ, which agreed with mixed inhibition indicating a preferential binding to the substrate-bound complex.

TABLE 1
The effect of compound BG-1 on the activity, conformational stability,
and limited tryptic proteolysis of HMBS

| | | Relative | | Protection |
|---|---|---|---|---|
| Compound | | activity, | Relative activity, | against tryptic |
| ID | ΔTₘ^{a} (° C.) | standard^{b} | pre-inc. at 70° C^{c} | proteolysis^{d} |
| CTRL | — | 1.00 | 1.00 | — |
| BG-1 | 1.6 | 0.98 ± 0.06 | 1.01 ± 0.06 | ++** |

^{a}The thermal upshift values at y = 0.5 in scaled fluorescence curves (ΔTₘ) monitored by DSF. The average compound concentration in DSF screening was 122 μM (2% DMSO).
^{b}Activity assay performed at standard conditions, with 100 μM PBG at 37° C., 84 μM compound and 2% DMSO, which was added in all controls.
^{c}Assay including pre-incubation of HMBS with compound at 70° C., and subsequent standard activity assay, with 100 μM PBG at 37° C., 84 μM compound and 2% DMSO.
^{d}Symbols: ++, 10% remaining full-length HMBS relative to DMSO.
**p < 0.01 for significant protection against tryptic proteolysis compared with the DMSO control sample, calculated by two-sample student's t-test for equal variance.

TABLE 2
Steady-state enzyme kinetic parameters of HMBS in the presence
of compound BG-1.

| | | | Vₘₐₓ^{a} |
|---|---|---|---|
| | | K_{M}(PBG)^{a} | (nmol/ |
| ID | Structure | (μM) | min/mg) |
| BG-1 | 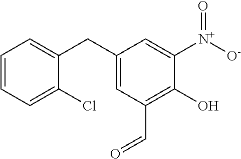 [SMILES: O=Cc1cc(Cc2ccccc2Cl)cc([N+](=O)[O-])c1O] | 69 ± 4* | 56 ± 1* |

^{a}Effect of the compound on the enzyme kinetic parameters for HMBS activity, measured at fixed compound concentration (84 μM in 2% DMSO) and variable PBG (0-1 mM) at 37° C., and fitted to Michaelis-Menten kinetics.
The K_{M} (PBG) and Vₘₐₓ values for the DMSO control of WT-HMBS were 86 ± 5 μM and 61 ± 2 nmol/min/mg, respectively.
*P ≤ 0.05, for significant difference compared with the values for DMSO control sample, calculated by unpaired two-tailed t-test.

### Example 16—Effect of Compound BG-1 in Human Hepatoma HepG2 Cells

The stabilizing and potential PC effect of compound BG-1 was investigated in HepG2 cells over-expressing HMBS by analyzing the compound concentration effect on the steady-state levels of the enzyme (see FIG. 2A). Quantitative western immunoblotting revealed an increasing relative amount of HMBS with increasing concentration of compound BG-1.

### Example 17—ALA Excretion in Hmbs-Deficient Mice (Trial T1/T2 A)

The compound heterozygote Hmbs-deficient T1/T2^{-/-} mouse model for AIP, which allows monitoring the level of precursors ALA and PBG in urine after phenobarbital induction, was used. One group of six mice was given 10 mg/kg/day (trial denoted T1/T2-A) of compound BG-1 for 12 days. A control group of six mice, treated with only DMSO was also included. To induce the heme synthesis, phenobarbital (Gardenal®) was given during the last three days of the study (see FIG. 3). No apparent toxicity in the treated mice was detected as assessed by normal behavior and organ appearances.

Hmbs-deficient T1/T2^{-/-} mice do not show elevated excretion of urinary ALA and PBG until induction of biochemical acute attacks (Lindberg et al., Nature genetics. 1996; 12(2):195-9), and indeed a rapid increase in urinary ALA and PBG was seen for the non-treated control mice by day 11 and even higher by day 12, following the administration of phenobarbital (white bars, FIG. 4A,B). The treatment with 10 mg/kg/day did not cause any significant change in HMBS protein levels or activity in either erythrocytes or liver, compared to the non-treated Hmbs-deficient mice in the T1/T2-A trial. However, a slight decreasing tendency in urinary levels of ALA, but not PBG, was observed for compound BG-1 treatment by day 12 (blue bars, FIG. 4B) compared to non-treated mice, indicating that a higher compound concentration may result in an increased metabolite level correction. Similarly, compound analogues with higher affinity might increase the effect due to more efficient dose-dependent effect in vivo. No toxic effect was registered for this compound.

### Example 18—Effect of Analogues of Compound BG-1 on the Thermal Stability, Proteolysis and Activity of HMBS

Compounds BG-2, BG-3 and BG-4 were tested on recombinant WT-HMBS using DSF and tryptic proteolysis. In cells, BG-2 increased the HMBS protein levels similarly to BG-1 (see FIGS. 2A and 2B), and enzyme kinetic analyses showed a weak mixed inhibitory effect (see Table 3).

TABLE 3
The effect of the compounds on the ΔTₘ measured by DSF, the limited tryptic
proteolysis and the activity of HMBS

| | | ΔTₘ^{b} | Protection against | K_{M}(PBG)^{d} | Vₘₐₓ^{d} |
|---|---|---|---|---|---|
| ID | Mw | (° C.) | tryptic proteolysis^{c} | (μM) | (nmol/min/mg) |
| CTRL | — | — | — | 86 ± 5 | 61 ± 2 |
| BG-2 | 261.66 | 4.9 | +* | 84 ± 8 | 54 ± 2** |
| 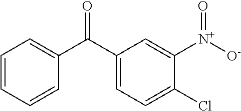 | | | | | |
| BG-3 | 257.24 | 6.6 | +/− | — | — |
| 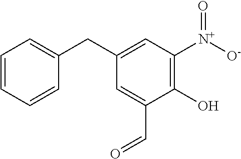 | | | | | |
| BG-4 | 293.7 | 4.7 | +/− | — | — |
| 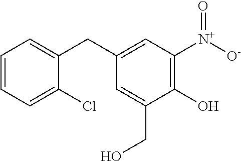 | | | | | |

^{b}The thermal upshift values (ΔTₘ) monitored by DSF. The average compound concentration in DSF screening was 122 μM (2% DMSO).
^{c}Symbols: +/−, ±2%; +, >4%.
^{d}Effect of the compounds on the enzyme kinetic parameters for HMBS activity, measured at fixed compound concentration (84 μM with 2% DMSO) and variable PBG (0-1 mM) at 37° C., and fitted to Michaelis-Menten kinetics.
*P < 0.05 and **p < 0.01, for significant difference compared with the values for DMSO control sample, calculated by unpaired two-tailed t-test.

### Example 19—Surface Plasmon Resonance (SPR) and Octet RED96 Studies

The binding of compounds BG-1 and BG-2 to HMBS was analyzed by surface plasmon resonance (SPR) and response units from concentration-dependent steady state measurements were analyzed assuming a 1:1 binding model. Compound BG-1 showed some unspecific binding, and an accurate S_{0.5} value could not be obtained. The interaction between compound BG-1 and HMBS was therefore further studied with Octet RED96 system with super streptavidin (SSA) biosensors. For the loading of the sensors, the protein needed to be biotinylated but no alteration of the buffer conditions was required. The data analysis using double reference subtraction accounts for non-specific binding and minimizes the well-based and sensor variability. The analyses with Octet provided an S_{0.5} value of 83±7 μM for compound BG-1, obtained by fitting the data to a (sigmoidal) binding isotherm with saturable concentration dependence (FIG. 5A (inset)). For compound BG-2, SPR allowed the measurement of good concentration-dependent binding data, which also yielded a sigmoidal binding curve, providing a S_{0.5} value of 63±3 μM (FIG. 5B).

### Example 20—Preventive Effect on ALA/PBG Excretion in Hmbs-Deficient Mice (Trial T1/T2-B)

In a second animal trial using Hmbs-deficient T1/T2^{-/-} mice, denoted T1/T2-B (n=6 in each group), the chaperone potential of compounds BG-1 and BG-2 at higher concentration (20 mg/kg/day) was investigated. The experimental setup was as for T1/T2-A (see FIG. 3). The experimental set up was otherwise identical, with n=6 in each treatment group and a control group (n=6) receiving DMSO instead of the compound. The effect of the compounds was, as in trial T1/T2-A, monitored by measuring urinary excretion of ALA and PBG. Both compound BG-1 and BG-2 reduced the urinary ALA and PBG excretion, and the latter to almost half of the value in the control group (FIG. 6A,B).

Quantitative western immunoblotting of liver tissue revealed a pronounced increase (2-fold) in steady-state levels of HMBS in the presence of the compounds BG-1 and BG-2 (FIG. 6C).

The effect of treatment with compound BG-1 and BG-2 on hepatic HMBS activity was measured in the liver homogenates, resulting in significantly increased enzyme activity in mice treated with both compounds as compared with the control group (FIG. 6D). Furthermore, the relative concentrations of ALA and PBG, as well as of compound accumulated in liver, which were found to be decreased for the mice treated with compound BG-1 (p<0.05 and p=0.053, respectively, compared with control mice; FIG. 4E,F).

### Example 21—Octet RED Studies on Other Compounds

Octet RED (Forte Bio) was used for screening binding and determining the K_{D} values of various compounds. OctetRED is based on the technique called Bio-layer interferometry (BLI). It measures changes in an interference pattern generated from visible light reflected from an optical layer and a biolayer containing proteins of interest. In the assay the target protein is biotinylated and coupled as a layer on the optic probe (Super Strepavidin). The concentration series was from 6.25 to 500 μM.

For the analysis method of K_{D} determination, steady state analysis was used, where the response from steady state of the association phase was used for determining the binding curve for the analyte. Buffer for the analysis was selected to PBS-P+5% DMSO.

TABLE 4
K_{D} values for the binding of the indicated compounds to HMBS,
measured by Octet.

| | K_{D} |
|---|---|
| Structure | (μM) |
| 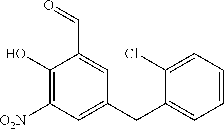 [SMILES: O=Cc1cc(Cc2ccccc2Cl)cc([N+](=O)[O-])c1O] | 90 |
| 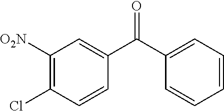 [SMILES: O=C(c1ccccc1)c1ccc(Cl)c([N+](=O)[O-])c1] | 40 |
| | 42 |
| | 160 |
| | 140 |
| | 400 |
| | mM range (no |
| | saturation with |
| | 500 μM) |
| | mM range (no |
| | saturation with |
| | 500 μM) |
| 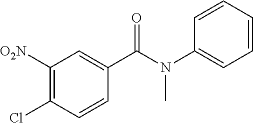 [SMILES: CN(C(=O)c1ccc(Cl)c([N+](=O)[O-])c1)c1ccccc1] | mM range (no saturation with 500 μM) |
| 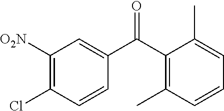 [SMILES: Cc1cccc(C)c1C(=O)c1ccc(Cl)c([N+](=O)[O-])c1] | mM range (no saturation with 500 μM) |
| 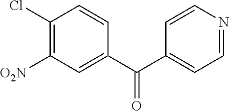 [SMILES: O=C(c1ccncc1)c1ccc(Cl)c([N+](=O)[O-])c1] | mM range (no saturation with 500 μM) |
| 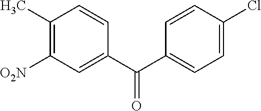 [SMILES: Cc1ccc(C(=O)c2ccc(Cl)cc2)cc1[N+](=O)[O-]] | 7 |
| 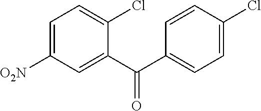 [SMILES: O=C(c1ccc(Cl)cc1)c1cc([N+](=O)[O-])ccc1Cl] | 110 |
| 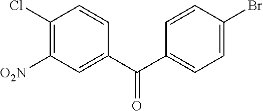 [SMILES: O=C(c1ccc(Br)cc1)c1ccc(Cl)c([N+](=O)[O-])c1] | mM range (no saturation with 500 μM) |
| 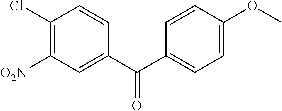 [SMILES: COc1ccc(C(=O)c2ccc(Cl)c([N+](=O)[O-])c2)cc1] | 300 |
| 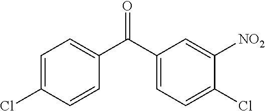 [SMILES: O=C(c1ccc(Cl)cc1)c1ccc(Cl)c([N+](=O)[O-])c1] | 200 |
| 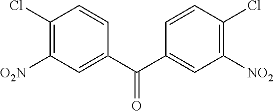 [SMILES: O=C(c1ccc(Cl)c([N+](=O)[O-])c1)c1ccc(Cl)c([N+](=O)[O-])c1] | mM range (no saturation with 500 μM) |
| 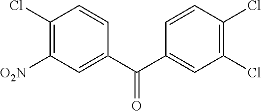 [SMILES: O=C(c1ccc(Cl)c(Cl)c1)c1ccc(Cl)c([N+](=O)[O-])c1] | 140 |
| 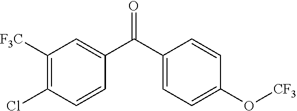 [SMILES: O=C(c1ccc(OC(F)(F)F)cc1)c1ccc(Cl)c(C(F)(F)F)c1] | 9.6 |
| 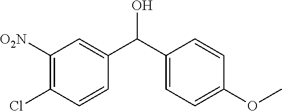 [SMILES: COc1ccc(C(O)c2ccc(Cl)c([N+](=O)[O-])c2)cc1] | >200 |
| 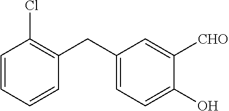 [SMILES: O=Cc1cc(Cc2ccccc2Cl)ccc1O] | 190 |
| 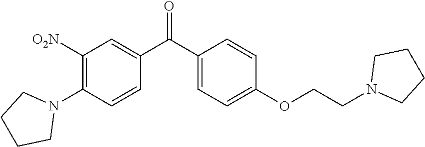 [SMILES: O=C(c1ccc(OCCN2CCCC2)cc1)c1ccc(N2CCCC2)c([N+](=O)[O-])c1] | >200 |

### Example 22—Further Octet RED Studies

Octet RED96 (Forte Bio) was used for screening binding and determining the K_{D} values for certain compounds. In the assay the target protein (WT-HMBS) was biotinylated and coupled as a layer on the optic probe. The concentration series for the tested compounds were from 3.1 to 150 μM, dissolved in PBS-P+5% DMSO.

For the analysis method of K_{D} determination, steady state analysis was used in which the response from steady-state of the association phase was used for determining the binding curve for the analyte. K_{D} values were calculated using sigmoidal fitting. The buffer for the analysis was selected to PBS-P+5% DMSO.

TABLE 5
K_{D} values for the binding of the indicated compounds to HMBS,
measured by Octet.

| | Soluble in | | | |
|---|---|---|---|---|
| | DMSO | Solubility in | | K_{D} |
| Structure | (100%) | buffer | Binding | (μM) |
| 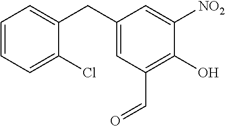 [SMILES: O=Cc1cc(Cc2ccccc2Cl)cc([N+](=O)[O-])c1O] | Yes | 384 μM | Yes | 83 |
| 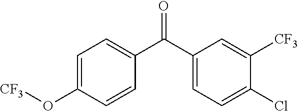 [SMILES: O=C(c1ccc(OC(F)(F)F)cc1)c1ccc(Cl)c(C(F)(F)F)c1] | Yes | 52 μM | Yes | 9.6 |
| 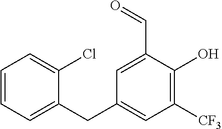 [SMILES: O=Cc1cc(Cc2ccccc2Cl)cc(C(F)(F)F)c1O] | Yes | Yes | Yes | 9.3 |
| 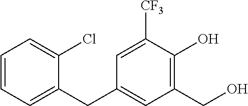 [SMILES: OCc1cc(Cc2ccccc2Cl)cc(C(F)(F)F)c1O] | Yes | Yes | Yes | 90 |

### Example 23—Activity Measurements In Vitro

Activity measurements were carried out using TECAN plate reader for absorbance measurements. Activity measurements were based on measuring the light absorbance at 405 nm for determining the concentration of formed product, preuroporphyrinogen. Standard curve for measurements was measured using absorbance of known concentrations for Uroporphyrin I dihydrochloride, the cyclic derivative of preuroporphyrinogen. Two concentrations of the compound of Example 14, i.e. 5 and 50 μM in 2.5% DMSO were used. Protein was incubated 30 min in 37° C. with the compound, and mixed with MIX-buffer (50 mM HEPES pH 8, 2.5% DMSO) on Corning 96 well plate (clear bottom, half area, black). The solutions were pre-warmed to 37° C. prior to reaction. Reaction was started by adding PBG and after 5 min the reaction was stopped by adding the STOP solution (5 M HCl and 0.1% p-Benzoquinone mixed 1:1). Wells were covered and protected from light during the whole reaction. Results show activation of HMBS for the compound of Example 14 when compared to HMBS with 5% DMSO only.

1. A method of prevention or treatment of a disease caused by a mutation in the gene coding for hydroxymethylbilane synthase, said method comprising the step of administering to a patient in need thereof a pharmaceutically effective amount of a compound of formula (I), or a pharmaceutically acceptable salt or prodrug thereof: 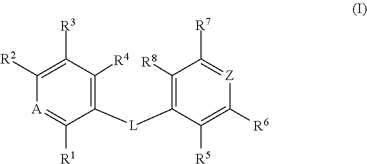
wherein:
A is selected from N and CR¹⁰ (wherein R¹⁰ is H, —NO₂, C₁₋₆ haloalkyl or —C(O)R¹⁷ in which R¹⁷ is H or C₁₋₆ alkyl);
Z is selected from N and CR⁹ (wherein R⁹ is H, halogen or —OR¹⁶ in which R¹⁶ is H, C₁₋₆ haloalkyl, or optionally substituted C₁₋₆ alkyl);
L is selected from —CH₂—, —C(O)—, —CH(OH)—, —C(O)—NR′—, and —NR′—C(O)— (wherein R′ is H or C₁₋₃ alkyl);
R¹ is H;
R² is selected from H, halogen, —NR¹¹R¹² (wherein R¹¹ and R¹² are independently selected from H and C₁₋₆ alkyl or, together with the nitrogen atom to which they are attached, form a 5- or 6-membered saturated ring), and —OR¹³ (wherein R¹³ is H or C₁₋₆ alkyl);
R³ is selected from H, —CH₂OH and —C(O)R¹⁴ (wherein R¹⁴ is H or C₁₋₆ alkyl);
R⁴ is selected from H, halogen and —OR¹⁵ (where R¹⁵ is H or C₁₋₆ alkyl);
R⁵ is selected from H and C₁₋₆ alkyl;
R⁶ is selected from H, —NO₂ and halogen;
R⁷ is H; and
R⁸ is selected from H, C₁₋₆ alkyl, and halogen;
or wherein:
R⁷ and R⁸ together with the intervening ring carbon atoms form an unsaturated ring.

2. The method according to claim 1, wherein R² is selected from H, halogen, and —OR¹³ (wherein R¹³ is H or C₁₋₆ alkyl).

3. The method according to claim 1, wherein R³ is selected from H and —C(O)H.

4. The method according to claim 1, wherein R⁴ is selected from H, —OH and Cl.

5. The method according to claim 1, wherein R⁶ is selected from H and halogen.

6. The method according to claim 1, wherein R⁸ is selected from H, halogen and —CH₃.

7. The method according to claim 1, wherein R⁷ and R⁸ together with the intervening ring carbon atoms form an unsaturated ring.

8. The method according to claim 1, wherein R⁹ is selected from H, halogen and —OR¹⁶ (wherein R¹⁶ is H, —CF₃ or —CH₃).

9. The method according to claim 1, wherein R¹⁰ is selected from —NO₂ and —CF₃.

10. The method according to claim 1, wherein the compound is a compound of general formula (IV), or a pharmaceutically acceptable salt or prodrug thereof: 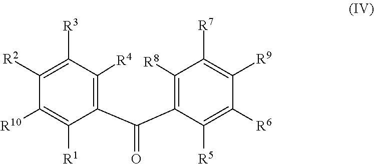
wherein R¹ to R¹⁰ are as defined in claim 1.

11. The method according to claim 1, wherein the compound is a compound of general formula (V), or a pharmaceutically acceptable salt or prodrug thereof: 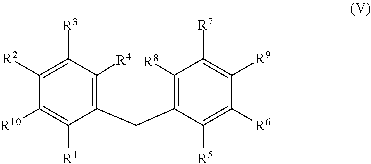
wherein R¹ to R¹⁰ are as defined in claim 1.

12. The method according to claim 1, wherein the compound is selected from the following and their pharmaceutically acceptable salts and prodrugs:

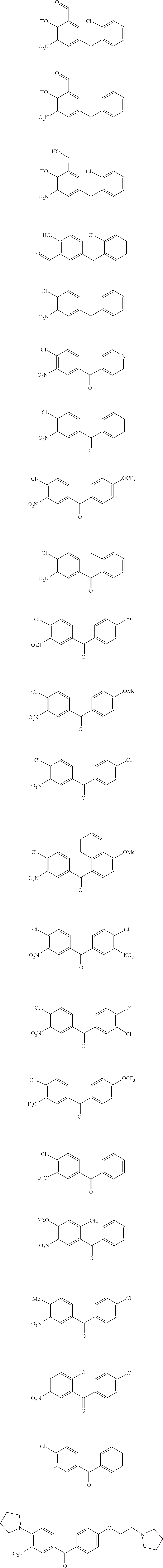

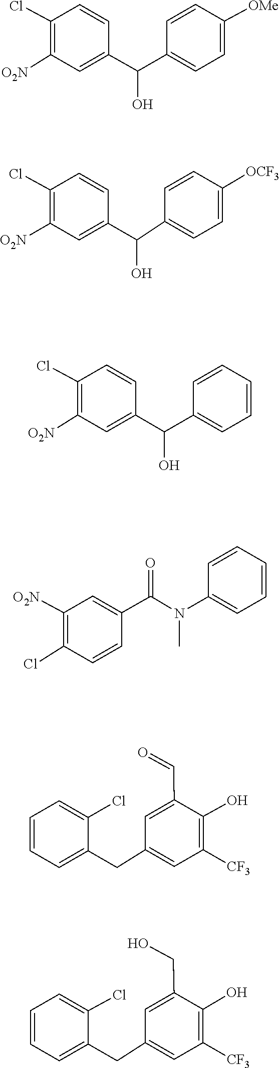 [SMILES: COc1ccc(C(O)c2ccc(Cl)c([N+](=O)[O-])c2)cc1 ; O=[N+]([O-])c1cc(C(O)c2ccc(OC(F)(F)F)cc2)ccc1Cl ; O=[N+]([O-])c1cc(C(O)c2ccccc2)ccc1Cl ; CN(C(=O)c1ccc(Cl)c([N+](=O)[O-])c1)c1ccccc1 ; O=Cc1cc(Cc2ccccc2Cl)cc(C(F)(F)F)c1O ; OCc1cc(Cc2ccccc2Cl)cc(C(F)(F)F)c1O]

13-24. (canceled)

25. The method according to claim 1, wherein the disease is acute intermittent porphyria.

26. The method according to claim 1, wherein R² is selected from H, —OCH₃, —OH and Cl.

27. The method according to claim 1, wherein R⁶ is selected from H and Cl.

28. The method according to claim 1, wherein R⁸ is selected from H and Cl.

29. The method according to claim 7, wherein the unsaturated ring is a 5- or 6-membered carbocyclic ring.

30. The method according to claim 7, wherein the unsaturated ring is an aryl ring.

31. The method according to claim 30, wherein the aryl ring is an optionally substituted phenyl ring.

32. The method according to claim 1, wherein R⁹ is selected from H, Cl, —OCF₃ and —OCH₃.
